(19) United States
(12) Patent Application Publication
(10) Pub. No.: US 2006/0154313 A1
(43) Pub. Date: Jul. 13, 2006

(54) Human B7 polypeptide B7-H3A

(75) Inventors: Dirk M. Anderson, Port Townsend, WA (US); John S. Marken, Seattle, WA (US)

Correspondence Address: IMMUNEX CORPORATION;LAW DEPARTMENT, 1201 AMGEN COURT WEST, SEATTLE, WA, 98119, US

(73) Assignee: Immunex Corporation, Thousand Oaks, CA (US)

(21) Appl. No.: 11/378,707

(22) Filed: Mar. 17, 2006

Related U.S. Application Data
(63) Continuation of application No. 10/294,830, filed on Nov. 14, 2002, now abandoned.
Application No. 10/294,830 is a continuation of application No. 09/835,045, filed on Apr. 13, 2001, now abandoned.
(60) Provisional application No. 60/196,967, filed on Apr. 13, 2000.

(51) Int. Cl. G01N 33/574; C07H 21/04; C12P 21/06; A61K 39/395; C07K 14/74; C07K 16/28

(52) U.S. Cl. 435/7.23; 435/69.1; 435/320.1; 435/325; 530/350; 530/388.22; 536/23.5; 424/143.1

(57) ABSTRACT

This invention relates to B7-H3A, a new member of the human B7 polypeptide family, methods of making such polypeptides, and to methods of using them to treat immunological conditions and to identify compounds that alter B7-H3A polypeptide activities.

This application is a continuation of application U.S. Ser. No. 10/294,830, filed Nov. 14, 2002, which is a continuation of application U.S. Ser. No. 09/835,045, filed Apr. 13, 2001; which claims the benefit under 35 U.S.C. § 119(e) of provisional application U.S. Ser. No. 60/196,967, filed Apr. 13, 2000; all of which are incorporated in their entirety by reference herein.

### FIELD OF THE INVENTION

This invention relates to B7-H3A (previously called ‘hh5336’), a new member of the human B7 polypeptide family, and to methods of making and using B7-H3A polypeptides.

### BACKGROUND OF THE INVENTION

Activation of T and B lymphocytes and the development of immune responses require specific antigen recognition by lymphocytes and additional costimulatory signals that are induced by non-self antigens, but not by self antigens. The B7 polypeptides are a related group of type I transmembrane polypeptides of the immunoglobulin (Ig) superfamily which serve as ligands for receptors on T cells and provide regulatory signals to T cells. When a T cell receives a “first signal” through its T cell receptor (TCR) interacting with an antigen-loaded MHC complex, the B7-1 (CD80) molecule sends an important co-stimulus or “second signal” to T cells through interaction with one of its two receptors, CD28. The B7-2 (CD86) molecule is upregulated on antigen-presenting cells by signals from the innate immune system or those delivered from activated T cells, such as through CD40L (CD 154); and like the B7-1 molecule, has been shown capable of delivering co-stimulation through CD28. Members of the B7 polypeptide family are expressed in a variety of cell types and can function at different stages in the development and regulation of T cell activity. For example, B7-1 and B7-2 each bind to both of the T cell receptors CD28 and CTLA4 and provide costimulatory signals to T cells, and B7-1 and B7-2 are both expressed by professional antigen-presenting cells such as dendritic cells, activated B cells, and macrophages, but B7-2 expression is upregulated more rapidly than B7-1 expression by the engagement of surface Ig molecules with antigen, producing a change over time in the ratio of B7-2 to B7-1 on the surface of antigen-presenting cells. Increased co-stimulation of T cells via the B7 molecules, for example, by transfection of B7-1 into tumor cells used as antigen, results in increased immune response against the tumor (Chambers and Allison, Curr Opin Cell Biol, 11(2):203-210, 1999). In vitro and in vivo evidence have demonstrated that this ability to provide an effective immune response is due to the cytokines produced by the T cells, most notably IL-2 to allow ag-specific T cell expansion, IL-4 to aid the humoral response, and IFN-gamma to arm the cytolytic arm of the adaptive immune response (McAdam et al, Immunol Rev, 165:231-47, 1998).

Additional B7 family polypeptides with other types of costimulatory activity have been described, such as B-7h (B7RP-1) and B7-H1 which provide a distinct co-stimulus to activated T cells that results in high IL-10 production with little IL-2 production (Swallow et al., Immunity, 11(4):423-432, 1999; Yoshinaga et al., Nature, 402:827-832, 1999; Dong et al., Nature Medicine, 5:1365-1369, 1999).

Common structural features of the B7 family of polypeptides are two Ig domains in the extracellular portion of these polypeptides: an N-terminal variable (V)-type Ig domain and a more membrane proximal constant (C)-type Ig domain. The extracellular domain is involved in binding to T cell receptors such as CD28, CTLA4, ICOS, and/or PD-1 to deliver a regulatory signal. Because of their roles in mediation of T cell immune response, B7 polypeptides are associated with immunological conditions such as the immune response to pathogens and cancer cells; transplant rejection and graft-versus-host disease (GVHD); allergies; and autoimmune diseases. For example, blocking the interaction of B7-1 and B7-2 polypeptides with a soluble form of one their binding partners, CTLA4, inhibited the progression of autoimmune disease in the non-obese diabetic (NOD) mouse and the mouse model for systemic lupus erythematosus (SLE or lupus). Characteristics and activities of the B7 polypeptide family are described further in the following references, which are incorporated by reference herein: Wang et al., 2000, Costimulation of T cells by B7-H2, a B7-like molecule that binds ICOS, Blood 96: 2808-2813; Freeman et al., 2000, Engagement of the PD-1 immunoinhibitory receptor by a novel B7 family member leads to negative regulation of lymphocyte activation, J Exp Med 192: 1027-1034; Yoshinaga et al., 2000, Characterization of a new human B7-related protein: B7RP-1 is the ligand to the co-stimulatory protein ICOS, Int Immunol 12: 1439-1447; Mages et al., 2000, Molecular cloning and characterization of murine ICOS and identification of B7h as ICOS ligand, Eur J Immunol 30: 1040-1047; Mueller DL, 2000, T cells: A proliferation of costimulatory molecules, Curr Biol 10: R227-R230; Ling et al., 2000, Cutting edge: identification of GL50, a novel B7-like protein that functionally binds to ICOS receptor, J Immunol 164: 1653-1657; Yoshinaga et al., 1999, T-cell co-stimulation through B7RP-1 and ICOS, Nature 402: 827-832; Dong et al., 1999, B7-H1, a third member of the B7 family, co-stimulates T-cell proliferation and interleukin-10 secretion, Nat Med 5: 1365-1369; Abbas and Sharpe, 1999, T-cell stimulation: an abundance of B7s, Nat Med 5: 1345-1346; Lenschow et al., 1996, CD28/B7 system of T cell costimulation, Annu Rev Immunol 14: 233-258; and Harlan et al., 1995, Potential roles of the B7 and CD28 receptor families in autoimmunity and immune evasion, Clin Immunol Immunopathol 75: 99-111.

In order to develop more effective treatments for immunological conditions and diseases, such as graft-versus-host disease and lupus, information is needed about previously unidentified members of the B7 polypeptide family.

### SUMMARY OF THE INVENTION

The present invention is based upon the discovery of a new human B7 family member, B7-H3A.

The invention provides an isolated polypeptide consisting of, consisting essentially of, or more preferably, comprising an amino acid sequence selected from the group consisting of:
(a) the amino acid sequence of SEQ ID NO:11;
(b) the amino acid sequence of SEQ ID NO:13;
(c) the amino acid sequence of SEQ ID NO:2;
(d) an amino acid sequence selected from the group consisting of:
   (d1) amino acids 27 through 169 of SEQ ID NO:2;
   (d2) amino acids 245 through 387 of SEQ ID NO:2;
   (d3) amino acids 29 through 387 of SEQ ID NO:2;
   (d4) amino acids 29 through 387 of SEQ ID NO:2 and further comprising amino acids 250 through 270 of SEQ ID NO:4;
   (d5) amino acids 29 through 387 of SEQ ID NO:2 and further comprising amino acids 273 through 316 of SEQ ID NO:4;
   (d6) amino acids Xaa1 through 139 of SEQ ID NO:11, wherein Xaa1 is an amino acid selected from the group consisting of amino acids 27 through 29 of SEQ ID NO:11;
   (d7) amino acids 140 through Xaa2 of SEQ ID NO:11, wherein Xaa2 is an amino acid selected from the group consisting of amino acids 238 through 244 of SEQ ID NO:11;
   (d8) amino acids Xaa3 through 357 of SEQ ID NO:11, wherein Xaa3 is an amino acid selected from the group consisting of amino acids 240 through 245 of SEQ ID NO:11;
   (d9) amino acids 358 through Xaa4 of SEQ ID NO:11, wherein Xaa4 is an amino acid selected from the group consisting of amino acids 456 through 465 of SEQ ID NO:11;
   (d10) amino acids Xaa5 through 534 of SEQ ID NO:11, wherein Xaa5 is an amino acid selected from the group consisting of amino acids 488 through 490 of SEQ ID NO:11;
(e) fragments of the amino acid sequences of any of (a)-(d) comprising at least 20 contiguous amino acids;
(f) fragments of the amino acid sequences of any of (a)-(d) having B7-H3A polypeptide activity;
(g) fragments of the amino acid sequences of any of (a)-(d) comprising B7-H3A extracellular domain amino acid sequences;
(h) amino acid sequences comprising at least 20 amino acids and sharing amino acid identity with the amino acid sequences of any of (a)-(g), wherein the percent amino acid identity is selected from the group consisting of: at least 70%, at least 75%, at least 80%, at least 85%, at least 90%, at least 95%, at least 97.5%, at least 99%, and at least 99.5%;
(i) an amino acid sequence of (h), wherein a polypeptide comprising said amino acid sequence of (h) binds to an antibody that also binds to a polypeptide comprising an amino acid sequence of any of (a)-(g);
(j) an amino acid sequence of (h) or (i) having B7-H3A polypeptide activity; and
(k) allelic variants of (a)-O) above.

Other aspects of the invention are isolated nucleic acids encoding polypeptides of the invention, with a preferred embodiment being an isolated nucleic acid consisting of, consisting essentially of, or more preferably, comprising a nucleotide sequence selected from the group consisting of:
(a) SEQ ID NO:1;
(b) SEQ ID NO:10;
(c) SEQ ID NO:12;
(d) a nucleotide sequence encoding an amino acid sequence selected from the group consisting of:
   (d1) amino acids 27 through 169 of SEQ ID NO:2;
   (d2) amino acids 245 through 387 of SEQ ID NO:2;
   (d3) amino acids 29 through 387 of SEQ ID NO:2;
   (d4) amino acids 29 through 387 of SEQ ID NO:2 and further comprising amino acids 250 through 270 of SEQ ID NO:4;
   (d5) amino acids 29 through 387 of SEQ ID NO:2 and further comprising amino acids 273 through 316 of SEQ ID NO:4;
   (d6) amino acids Xaa1 through 139 of SEQ ID NO:11, wherein Xaa1 is an amino acid selected from the group consisting of amino acids 27 through 29 of SEQ ID NO:11;
   (d7) amino acids 140 through Xaa2 of SEQ ID NO:11, wherein Xaa2 is an amino acid selected from the group consisting of amino acids 238 through 244 of SEQ ID NO:11;
   (d8) amino acids Xaa3 through 357 of SEQ ID NO:11, wherein Xaa3 is an amino acid selected from the group consisting of amino acids 240 through 245 of SEQ ID NO:11;
   (d9) amino acids 358 through Xaa4 of SEQ ID NO:11, wherein Xaa4 is an amino acid selected from the group consisting of amino acids 456 through 465 of SEQ ID NO:11;
   (d10) amino acids Xaa5 through 534 of SEQ ID NO:11, wherein Xaa5 is an amino acid selected from the group consisting of amino acids 488 through 490 of SEQ ID NO:11; and
(e) allelic variants of (a)-(d).

The invention also provides isolated genomic nucleic acids corresponding to the nucleic acids of the invention.

Other aspects of the invention are isolated nucleic acids encoding polypeptides of the invention, and isolated nucleic acids, preferably having a length of at least 15 nucleotides, that hybridize under conditions of moderate stringency to the nucleic acids encoding polypeptides of the invention. In preferred embodiments of the invention, such nucleic acids encode a polypeptide having B7-H3A polypeptide activity, or comprise a nucleotide sequence that shares nucleotide sequence identity with the nucleotide sequences of the nucleic acids of the invention, wherein the percent nucleotide sequence identity is selected from the group consisting of: at least 70%, at least 75%, at least 80%, at least 85%, at least 90%, at least 95%, at least 97.5%, at least 99%, and at least 99.5%.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. A preferred process provided by the invention further comprises purifying said polypeptide. In another aspect of the invention, the polypeptide produced by said process is provided.

Further aspects of the invention are isolated antibodies that bind to the polypeptides of the invention, preferably monoclonal antibodies, also preferably humanized antibodies or humanized antibodies, and preferably wherein the antibody inhibits the activity of said polypeptides.

The invention additionally provides a method of designing an inhibitor of the polypeptides of the invention, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

In a further aspect of the invention, a method is provided for identifying compounds that alter B7-H3A polypeptide activity comprising
(a) mixing a test compound with a polypeptide of the invention; and
(b) determining whether the test compound alters the B7-H3A polypeptide activity of said polypeptide.

In another aspect of the invention, a method is provided identifying compounds that inhibit the binding activity of B7-H3A polypeptides comprising
(a) mixing a test compound with a polypeptide of the invention and a binding partner of said polypeptide; and
(b) determining whether the test compound inhibits the binding activity of said polypeptide.
   In preferred embodiments, the binding partner is a member of the Ig superfamily of polypeptides; more preferably, the binding partner is a T cell receptor polypeptide; and most preferably, the binding partner shares significant sequence similarity with CD28, CTLA4, ICOS, and/or PD-1.

The invention also provides a method for increasing T cell activities, comprising providing at least one compound selected from the group consisting of the polypeptides of the invention and agonists of said polypeptides; with a preferred embodiment of the method further comprising increasing said activities in a patient by administering at least one polypeptide of the invention.

Further provided by the invention is a method for decreasing T cell activities, comprising providing at least one antagonist of the polypeptides of the invention; with a preferred embodiment of the method further comprising decreasing said activities in a patient by administering at least one antagonist of the polypeptides of the invention, and with a further preferred embodiment wherein the antagonist is an antibody that inhibits the activity of any of said polypeptides.

The invention additionally provides a method for treating an immunological condition comprising administering at least one compound selected from the group consisting of the polypeptides of the invention and agonists of said polypeptides; with a preferred embodiment wherein the immunological condition is a T cell related condition, and/or is selected from the group consisting of cancer, including metastasis of cancer cells; bacterial or viral infections, including HIV infection; delayed reconstitution of T cells, for example following bone marrow transplantation; defects in T cell or accessory cell function, for example in hemodialysis patients subject to renal failure; and congenital immunodeficiencies.

In other aspects of the invention, a method is provided for treating an immunological condition comprising administering an antagonist of the polypeptide of the invention; with a preferred embodiment wherein the immunological condition is a T cell related condition, and/or is selected from the group consisting of transplant rejection; graft-versus-host disease; allergy; asthma; inflammatory bowel disease (IBD); sepsis; diseases that are caused or exacerbated by T cell mediated inflammation, such as Alzheimer's disease and atherosclerosis; and autoimmune diseases such as systemic lupus erythematosus (SLE or lupus), Grave's disease, psoriasis, autoimmune demyelination, multiple sclerosis, autoimmune diabetes and diabetic neuropathy, and rheumatoid arthritis.

A further embodiment of the invention provides a use for the polypeptides of the invention and agonsits thereof in the preparation of a medicament for treating an immunological condition; with a preferred embodiment wherein the immunological condition is cancer, including metastasis of cancer cells; bacterial or viral infections, including HIV infection; delayed reconstitution of T cells, for example following bone marrow transplantation; defects in T cell or accessory cell function, for example in hemodialysis patients subject to renal failure; and congenital immunodeficiencies. Also provided as an aspect of the invention is a use for antagonists of the polypeptides of the invention in the preparation of a medicament for treating an immunological condition; with a preferred embodiment wherein the immunological condition is transplant rejection; graft-versus-host disease; allergy; asthma; inflammatory bowel disease (IBD); sepsis; diseases that are caused or exacerbated by T cell mediated inflammation, such as Alzheimer's disease and atherosclerosis; and autoimmune diseases such as systemic lupus erythematosus (SLE or lupus), Grave's disease, psoriasis, autoimmune demyelination, multiple sclerosis, autoimmune diabetes and diabetic neuropathy, and rheumatoid arthritis.

In another embodiment of the invention, a use is provided for the polypeptides of the invention and/or agonists thereof as an adjuvant, for increasing the immunogenic effectiveness of an immunogenic preparation or vaccine, and/or for increasing the production of Th1 cells or increasing the proportion of T cells that differentiate into Th1 cells, and in the preparation of a medicament for such uses.

In another embodiment of the invention, a use is provided for antagonists of polypeptides of the invention as an adjuvant for increasing the production of Th2 cells or increasing the proportion of T cells that differentiate into Th2 cells, and in the preparation of a medicament for such use.

### DETAILED DESCRIPTION OF THE INVENTION

Similarities of B7-H3A Structure to Other B7 Family Members

We have identified B7-H3A, a new human B7 polypeptide having structural features characteristic of this polypeptide family; the amino acid sequence of a B7-H3A polypeptide is provided in SEQ ID NO:11 and an alignment showing the sequence similarities between B7-H3A and other B7 polypeptides is presented in Table 1 in Example 1 below. A naturally occuring variant of the B7-H3A has also been identified and is referred to herein as the B7-H3A ‘8b’ variant; its amino acid is provided in SEQ ID NO:13. The amino acid substitutions present in B7-H3A ‘8b’ relative to the B7-H3A polypeptide of SEQ ID NO:11 are likely due to allelic variation present within the human population. B7-H3A polypeptide is similar to a human polypeptide recently described in the literature and called B7 homolog 3, or “B7-H3” (see Chapoval et al., 2001, Nature Immunol 2: 269-274 and Table 2 below); however, B7-H3A contains four Ig domains rather than the two Ig domains described for B7-H3. Like B7-H3, B7-H3A polypeptide is expressed on dendritic cells, which are the first cells to activate naïve T cells, and is likely to modulate T cell activity by binding to one or more T cell receptors and providing a costimulatory signal, resulting in increased levels of interferon gamma (IFN-gamma) by T cells. This pattern of cytokine production is consistent with B7-H3A inducing an increase in the differentiation of precursor T cells into Th1 cells that produce IFN-gamma and IL-2 and mediate cellular immune responses. The receptors on T cells for B7 family polypeptides include CD28, CTLA4, PD-1, and ICOS; the receptor(s) for B7-H3A polypeptide are likely to be members of this subset of the Ig superfamily. B7-H3A polypeptides may modulate T cell activity by delivering a direct costimulatory signal to T cells by binding receptor molecules on those T cells. Alternatively, B7-H3A polypeptides may act by binding to receptor molecules on T cells, altering the cytokines secreted by those T cells, which in turn alters the T-cell-regulating and costimulatory activities of antigen presenting cells present at or recruited to the site. A combination of direct costimulatory effects on T cells and the effect of altered T cell cytokine secretion on multiple antigen presenting cells (and through them, multiple T cells) is believed to provide the network of regulatory cell-cell interaction that results in appropriate levels of immune activity against non-self antigens.

Structural elements common to members of the B7 polypeptide family include an extracellular domain which typically includes one V-like and one C-like Ig domain. A signal sequence is found at the N-terminus of full-length B7 family polypeptides, and is followed, in N-to-C order, by a V-like Ig domain, a C-like Ig domain, a transmembrane domain, and an intracellular domain. The B7-H3A polypeptide has a signal sequence extending from amino acid 1 to approximately amino acid 28 of SEQ ID NO:11, with the mature polypeptide produced by cleavage of the signal sequence predicted to have an amino acid sequence beginning at amino acid 29 of SEQ ID NO:11. The B7-H3A polypeptide has an N-terminal V-like Ig domain (“Ig domain 1”) extending from approximately between amino acid 27 and amino acid 29 to approximately amino acid 139 of SEQ ID NO:11; a C-like Ig domain (“Ig domain 2”) extending from approximately amino acid 140 to approximately between amino acid 238 and amino acid 244 of SEQ ID NO:11; a second V-like Ig domain (“Ig domain 3”) extending from approximately between amino acid 240 and amino acid 245 to approximately amino acid 357 of SEQ ID NO:11; a second C-like Ig domain (“Ig domain 4”) extending from approximately amino acid 358 to approximately between amino acid 456 and amino acid 465 of SEQ ID NO:11; a transmembrane domain extending from approximately between amino acid 466 and amino acid 468 to approximately between amino acid 487 and amino acid 489 of SEQ ID NO:11; and a cytoplasmic domain extending from the end of the transmembrane domain (i.e. beginning roughly between amino acid 488 and amino acid 490OF SEQ ID NO:11) and extending through the carboxyl terminus of the polypeptide (amino acid 534 of SEQ ID NO:11). Therefore, B7-H3A polypeptide has an overall structure consistent with other B7 polypeptides, but is distinct in containing four Ig domains.

The extracellular domain of B7 polypeptides extends from the N-terminus to the transmembrane domain (i.e. from approximately amino acid 29 through between amino acid 465 and 467 of SEQ ID NO:11), and includes the two sets of V-like and C-like Ig domains, i.e. Ig domains 1-4. There are certain key residues within the extracellular domains of B7 polypeptides, the two pairs of conserved cysteine residues—one pair in each Ig domain—that are involved in disulfide bond formation and the three-dimensional conformation of the polypeptide, such that substitutions of those residues are likely be associated with an altered function or lack of that function for the polypeptide. The conserved cysteines within the B7-H3A polypeptide are located at amino acid positions 50, 122, 165, 220, 268, 340, 383, and 438 of SEQ ID NO:11. The intracellular domain of B7 polypeptides extends from the transmembrane domain to the C terminus. Although in the preceeding examples the locations of the polypeptide domains have been given with respect to the amino acid sequence of SEQ ID NO:11, these domains are found at the same locations within the B7-H3A ‘8b’ polypeptide of SEQ ID NO:13, and for the signal sequence through the middle of Ig domain 3, for the the B7-H3A ‘hh5336’ polypeptide of SEQ ID NO:2 as well. The skilled artisan will recognize that the boundaries of the regions of B7-H3A polypeptides described above are approximate and that the precise boundaries of such domains, as for example the boundaries of the transmembrane region (which can be predicted by using computer programs available for that purpose), can also differ from member to member within the B7 polypeptide family.

The B7 polypeptide family is moderately conserved, with the Ig domains of human family members very similar to each other, and to the Ig domains of B7 family members from other species such as Mus musculus, Canis familiaris, Felis catus, and Sus scrofa, but are poorly conserved outside of the Ig domains. However, subfamilies of the B7 polypeptide family can be defined on the basis of presence of an intracellular B30.2 domain. These subfamilies are generally referred to as the immunomodulatory B7 family members, which include B7-1 (CD80), B7-2 (CD86), and B7-H1, and the butyrophilin (BTN)/MOG (myelin oligodendrocyte glycoprotein-like) family members, with the immunomodulatory B7 subfamily lacking a B30.2 domain and the butyrophilin/MOG subfamily having a B30.2 domain. As the B7-H3A polypeptide lacks an intracellular B30.2 domain, it is most similar to the immunomodulatory B7 family members, B7-H3 in particular, and is therefore considered a member of this B7 polypeptide subgroup.

Biological Activities and Functions of B7-H3A Polypeptides

Typical biological activities or functions associated with the B7 family of polypeptides are T cell costimulation in the case of the immunomodulatory B7 family members, and MHC molecule functions, such as regulating the antigen specificity of T lymphocyte responses, in the case of the MHC-encoded butyrophilin/MOG B7 subfamily members (see, for example, Stefferl et al., 2000, Butyrophilin, a milk protein, modulates the encephalitogenic T cell response to myelin oligodendrocyte glycoprotein in experimental autoimmune encephalomyelitis, J. Immunol. 165: 2859-2865). B7-H3A polypeptides having T cell immunomodulatory activity bind to T cell receptor molecules. The T cell immunomodulatory activity is associated with the extracellular domain of B7-H3A polypeptides. Thus, for uses requiring T cell immunomodulatory activity, preferred B7-H3A polypeptides include those having the extracellular domain and exhibiting T cell immunomodulatory biological activity. In addition, the extracellular domains of B7-H3A polypeptides are likely to be involved in any “reverse signaling” of dendritic cells by binding partners of B7-H3A polypeptides, that is, transmission of a an extracellular signal from the extracellular domain of a B7-H3A polypeptide to the intracellular domain of such polypeptide, this signal modulating dendritic cell activity, proliferation, and/or development. Preferred B7-H3A polypeptides therefore further include oligomers or fusion polypeptides comprising at least one extracellular portion of one or more B7-H3A polypeptides, and fragments of any of these polypeptides that have T cell immunomodulatory activity or are required for modulation of dendritic cell activity. Particularly preferred embodiments of the invention are B7-H3A polypeptides comprising the N-terminal V-like and C-like Ig domains (i.e. Ig domains 1 and 2 or approximately amino acids 29-238) of the B7-H3A polypeptides of SEQ ID Nos 2, 11, and 13; and B7-H3A polypeptides comprising the N-terminal V-like Ig domain (i.e. Ig domain 1 or approximately amino acids 29-139) of the B7-H3A polypeptides of SEQ ID Nos 2, 11, and 13. In further preferred embodiments, such extracellular Ig domains are fused to Fc molecules. As one example of such polypeptides, an amino acid sequence comprising the N-terminal V-like and C-like Ig domains (i.e. Ig domains 1 and 2 or approximately amino acids 29-238) of SEQ ID NO:13 fused to an Fc domain is provided as SEQ ID NO:20.

The T cell immunomodulatory activity of B7-H3A polypeptides can be determined, for example, by measuring the change in ³H-thymidine uptake or in cytokine secretion (such as IFN gamma secretion) by T cells exposed to surface-bound or soluble B7-H3A polypeptide (see, for example, FIGS. 3 through 5 of Dong et al., 1999, B7-H1, a third member of the B7 family, co-stimulates T-cell proliferation and interleukin-10 secretion, Nat Med 5: 1365-1369). The term “B7-H3A polypeptide activity,” as used herein, includes any one or more of the following: T cell immunomodulatory activity (the ability to regulate or modulate T cell activity, including T cell costimulation activity), the regulation of T cell costimulation activity by modulating the effects of T cells on antigen-presenting cells, and modulating the differentiation of precursor T cells to increase the ratio of Th1 cells to Th2 cells in the effector cells that are produced (also called “immune deviation” activity, as well as the ex vivo and in vivo activities of B7-H3A polypeptides. The degree to which B7-H3A polypeptides and fragments and other derivatives of these polypeptides exhibit these activities can be determined by standard assay methods. Exemplary assays are disclosed herein; those of skill in the art will appreciate that other, similar types of assays can be used to measure B7-H3A biological activities.

Another aspect of the biological activity of B7 polypeptides is the ability of members of this polypeptide family to bind particular binding partners, for example, T cell receptors such as CD28, CTLA4, ICOS, and/or PD-1, with the extracellular domain of the B7 polypeptide binding to the extracellular domain of the T cell receptor. The term “binding partner,” as used herein, includes ligands, receptors, substrates, antibodies, other B7 polypeptides, the same B7-H3A polypeptide (in the case of homotypic interactions), and any other molecule that interacts with a B7-H3A polypeptide through contact or proximity between particular portions of the binding partner and the B7-H3A polypeptide. A preferred binding partner for B7-H3A polypeptides is an Ig superfamily polypeptide, preferably a receptor expressed on T cells, and preferably having sequence similarity to the family of T cell receptors including as CD28, CTLA4, ICOS, and PD-1. The interactions between B7-H3A polypeptides and their binding partners are involved in mediating interactions between cell types including antigen presenting cells and T cells. Because the extracellular domain of B7-H3A polypeptides binds to T cell receptors, the extracellular domain when expressed as a separate fragment from the rest of a B7-H3A polypeptide, or as a soluble polypeptide, fused for example to an immunoglobulin Fc domain, is expected to disrupt the binding of B7-H3A polypeptides to their binding partners. By binding to one or more binding partners, the separate extracellular domain polypeptide likely prevents binding by the native B7-H3A polypeptide(s), and so acts in a dominant negative fashion to inhibit the biological activities mediated via binding of B7-H3A polypeptides to T cell receptors. Particularly suitable assays to detect or measure the binding between B7-H3A polypeptides and their binding partners are fluorescence-activated cell sorting (FACS) methods (see, for example, FIG. 1d of Dong et al., 1999, B7-H1, a third member of the B7 family, co-stimulates T-cell proliferation and interleukin-10 secretion, Nat Med 5: 1365-1369). Additional assays for evaluating the biological activities and partner-binding properties of B7-H3A family polypeptides are described below and in the references cited herein.

B7-H3A polypeptides are involved in immunological diseases or conditions, that share as a common feature T cell activity in their etiology. Blocking or inhibiting the interactions between members of the B7-H3A polypeptide family and their substrates, ligands, receptors, binding partners, and or other interacting polypeptides is an aspect of the invention and provides methods for treating or ameliorating the following diseases and conditions through the use of inhibitors of B7-H3A polypeptide activity: transplant rejection; graft-versus-host disease; allergy; asthma; inflammatory bowel disease (IBD); sepsis; diseases that are caused or exacerbated by T cell mediated inflammation, such as Alzheimer's disease and atherosclerosis; and autoimmune diseases such as systemic lupus erythematosus (SLE or lupus), Grave's disease, psoriasis, autoimmune demyelination, multiple sclerosis, autoimmune diabetes and diabetic neuropathy, and rheumatoid arthritis. Examples of such inhibitors or antagonists are described in more detail below. For certain conditions involving or exacerbated by absence of or a low level of B7-H3A polypeptide activity, such as: cancer, including metastasis of cancer cells; bacterial or viral infections, including HIV infection; delayed reconstitution of T cells, for example following bone marrow transplantation; defects in T cell or accessory cell function, for example in hemodialysis patients subject to renal failure; congenital immunodeficiencies; methods of treating or ameliorating these conditions comprise increasing the amount or activity of B7-H3A polypeptides by providing isolated B7-H3A polypeptides or active fragments or fusion polypeptides thereof, or by providing compounds (agonists) that activate endogenous or exogenous B7-H3A polypeptides.

Additional uses for B7-H3A polypeptides include diagnostic reagents for immunological diseases, research reagents for investigation of antigen presenting cell and T cell polypeptides and/or processes, purification/processing/preservation of antigen presenting cells or T cells, or as a carrier/targeting polypeptide to deliver therapeutic agents to T cells. Another use for polypeptides of the invention and agonists thereof is use as an adjuvant, for increasing the immunogenicity of an immunogenic preparation or vaccine and/or increasing the proportion of Th1 cells, and antagonists of the polypeptides of the invention may also be used as an adjuvant, for increasing the proportion of Th2 cells, as described in more detail below.

B7-H3A Polypeptides

A B7-H3A polypeptide is a polypeptide that shares a sufficient degree of amino acid identity or similarity to the B7-H3A polypeptides of SEQ ID Nos 2, 11, and/or 13 to (A) be identified by those of skill in the art as a polypeptide likely to share particular structural domains and/or (B) have biological activities in common with the B7-H3A polypeptide of SEQ ID Nos 2, 11, and/or 13 and/or (C) bind to antibodies that also specifically bind to other B7-H3A polypeptides. B7-H3A polypeptides can be isolated from naturally occurring sources, or have the same structure as naturally occurring B7-H3A polypeptides, or can be produced to have structures that differ from naturally occurring B7-H3A polypeptides. Polypeptides derived from any B7-H3A polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules) are also B7-H3A polypeptides. Therefore, the polypeptides provided by the invention include polypeptides characterized by amino acid sequences similar to those of the B7-H3A polypeptides described herein, but into which modifications are naturally provided or deliberately engineered. A polypeptide that shares biological activities in common with B7-H3A polypeptides is a polypeptide having B7-H3A polypeptide activity. Examples of biological activities exhibited by B7-H3A polypeptides include, without limitation, T cell immunomodulatory activity (the ability to regulate or modulate T cell activity, including T cell costimulation activity), the regulation of T cell costimulation activity by modulating the effects of T cells on antigen-presenting cells, and modulating the differentiation of precursor T cells to increase the ratio of Th1 cells to Th2 cells in the effector cells that are produced (also called “immune deviation” activity).

The present invention provides both full-length and mature forms of B7-H3A polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide can be obtained by expression, in a suitable mammalian cell or other host cell, of a nucleic acid molecule that encodes the full-length polypeptide. The sequence of the mature form of the polypeptide may also be determinable from the amino acid sequence of the full-length form, through identification of signal sequences or protease cleavage sites. The B7-H3A polypeptides of the invention also include those that result from post-transcriptional or post-translational processing events such as alternate mRNA processing which can yield a truncated but biologically active polypeptide, for example, a naturally occurring soluble form of the polypeptide. Also encompassed within the invention are variations attributable to proteolysis such as differences in the N- or C-termini upon expression in different types of host cells, due to proteolytic removal of one or more terminal amino acids from the polypeptide (generally from 1-5 terminal amino acids).

The invention further includes B7-H3A polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of B7-H3A polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of B7-H3A polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the B7-H3A polypeptides of the present invention are encompassed by the present invention and can be in linear form or cyclized using known methods, for example, as described in Saragovi et al., Bio/Technology 10, 773-778 (1992) and in McDowell et al., J Amer Chem Soc 114 9245-9253 (1992). Polypeptides and polypeptide fragments of the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of a B7-H3A polypeptide and have at least 60% sequence identity (more preferably at least 70%, at least 75%, at least 80%, at least 85%, at least 90%, at least 95%, at least 97.5%, or at least 99%, and most preferably at least 99.5%) with that B7-H3A polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any B7-H3A polypeptide, where sequence identity is determined by comparing the amino acid sequences of the polypeptides when aligned so as to maximize overlap and identity while minimizing sequence gaps. The percent identity can be determined by visual inspection and mathematical calculation, or the percent identity of two amino acid or two nucleic acid sequences can be determined by comparing sequence information using the GAP computer program, version 6.0 described by Devereux et al. (Nucl Acids Res 12: 387, 1984) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by those skilled in the art of sequence comparison can also be used, such as, for example, the BLASTN program version 2.0.9, available for use via the National Library of Medicine website: www.ncbi.nlm.nih.gov/gorf/wblast2.cgi, or the UW-BLAST 2.0 algorithm. Standard default parameter settings for UW-BLAST 2.0 are described at the following Internet site: http://blast.wustl.edu/blast/README.html#References. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton & Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Clayerie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The present invention also provides for soluble forms of B7-H3A polypeptides comprising certain fragments or domains of these polypeptides, and particularly those comprising the extracellular domain or one or more fragments of the extracellular domain. Soluble polypeptides are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble B7-H3A polypeptides also include those polypeptides which include part of the transmembrane region, provided that the soluble B7-H3A polypeptide is capable of being secreted from a cell, and preferably retains B7-H3A polypeptide activity. Soluble B7-H3A polypeptides further include oligomers or fusion polypeptides comprising the extracellular portion of at least one B7-H3A polypeptide, and fragments of any of these polypeptides that have B7-H3A polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of B7-H3A polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect of the invention, preferred polypeptides comprise various combinations of B7-H3A polypeptide domains, such as the N-terminal V-like Ig domain (Ig domain 1) and C-like Ig domain (Ig domain 2). Accordingly, polypeptides of the present invention and nucleic acids encoding them include those comprising or encoding two or more copies of a domain such as the V-like Ig domain 1, two or more copies of a domain such as the C-like Ig domain 2, or at least one copy of each domain, and these domains are preferably presented with a V-like Ig domain N-terminal to a C-like Ig domain where both such types of Ig domains are present within such polypeptides.

Further modifications in the peptide or DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in poly-peptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide (Asp-Tyr-Lys-Asp-Asp-Asp-Asp-Lys, SEQ ID NO:8), which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Encompassed by the invention are oligomers or fusion polypeptides that contain a B7-H3A polypeptide, one or more fragments of B7-H3A polypeptides, or any of the derivative or variant forms of B7-H3A polypeptides as disclosed herein. In particular embodiments, the oligomers comprise soluble B7-H3A polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple B7-H3A polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

In embodiments where variants of the B7-H3A polypeptides are constructed to include a membrane-spanning domain, they will form a Type I membrane polypeptide. Membrane-spanning B7-H3A polypeptides can be fused with extracellular domains of receptor polypeptides for which the ligand is known. Such fusion polypeptides can then be manipulated to control the intracellular signaling pathways triggered by the membrane-spanning B7-H3A polypeptide. B7-H3A polypeptides that span the cell membrane can also be fused with agonists or antagonists of cell-surface receptors, or cellular adhesion molecules to further modulate B7-H3A intracellular effects. In another aspect of the present invention, interleukins can be situated between the preferred B7-H3A polypeptide fragment and other fusion polypeptide domains.

Immunoglobulin-based Oligomers. The polypeptides of the invention or fragments thereof can be fused to molecules such as immunoglobulins for many purposes, including increasing the valency of polypeptide binding sites. For example, fragments of a B7-H3A polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four B7-H3A extracellular regions.

Peptide-linker Based Oligomers. Alternatively, the oligomer is a fusion polypeptide comprising multiple B7-H3A polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble B7-H3A polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for screening or other immunological methodologies can also be made by those skilled in the art given the disclosures herein. Such modifications are believed to be encompassed by the present invention.

Nucleic Acids Encoding B7-H3A Polypeptides

Encompassed within the invention are nucleic acids encoding B7-H3A polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a nucleic acid molecule encoding a B7-H3A polypeptide or a desired combination of B7-H3A polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990). Particularly preferred oligonucleotide probes for the present invention would include an oligonucleotide comprising the nucleic acid sequence of SEQ ID NO:5 (CACAGTTTCACCGAAGGCCGGGACCA) or, alternatively, for example, an oligonucleotide comprising the nucleic acid sequence TGAGGACCCGGTGGTGGCCCTAGT (SEQ ID NO:6). Such probes are particularly useful as they will hybridize to a nucleic acid encoding a B7-H3A polypeptide, but will distinguish it from other non-B7-H3-like molecules. An additional preferred oligonucleotide is shown as SEQ ID NO:7; this sequence encodes the six-amino acid sequence from amino acid 239 to amino acid 244 of SEQ ID Nos 2, 11, and 13, and because this six-amino acid sequence is situated between the repeated amino acid sequences of the B7-H3A polypeptides but is not present in either repeat, an oligonucleotide comprising SEQ ID NO:7 (or its reverse complement) can be used to uniquely PCR amplify either of the two repeat-encoding sequences within B7-H3A nucleic acids.

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the invention relates to certain isolated nucleic acids that are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also includes nucleic acids that hybridize under moderately stringent conditions, and more preferably highly stringent conditions, to nucleic acids encoding B7-H3A polypeptides described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1×SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10.degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (more preferably, 5000 basepairs, still more preferably, 2500 basepairs, and most preferably, 1000 basepairs) of genomic nucleic acid sequence upstream of the first nucleotide of the genomic sequence corresponding to the initiation codon of the B7-H3A coding sequence, and 10000 basepairs (more preferably, 5000 basepairs, still more preferably, 2500 basepairs, and most preferably, 1000 basepairs) of genomic nucleic acid sequence downstream of the last nucleotide of the genomic sequence corresponding to the termination codon of the B7-H3A coding sequence. The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or “an isolated genomic nucleic acid” is a genomic nucleic acid that has been separated from the adjacent genomic sequences present in the genome of the organism from which the genomic nucleic acid was isolated.

Methods for Making and Purifying B7-H3A Polypeptides

Methods for making B7-H3A polypeptides are described below. Expression, isolation, and purification of the polypeptides and fragments of the invention can be accomplished by any suitable technique, including but not limited to the following methods. Preferred host cells for producing recombinant B7-H3A polypeptides are CHO cells.

The isolated nucleic acid of the invention can be operably linked to an expression control sequence such as the pDC412 or pDC314 vectors, or the pMT2 or pED expression vectors disclosed in Kaufman et al., Nucleic Acids Res. 19, 4485-4490 (1991); and Pouwels et al. Cloning Vectors: A Laboratory Manual, Elsevier, N.Y., (1985), in order to produce the polypeptide recombinantly. Many suitable expression control sequences are known in the art. General methods of expressing recombinant polypeptides are also known and are exemplified in R. Kaufman, Methods in Enzymology 185, 537-566 (1990). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells promotes extracellular secretion of the polypeptide. The signal peptide is cleaved from the polypeptide upon secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells can act as suitable host cells for expression of the polypeptide. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. (EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it is possible to produce the polypeptide in lower eukaryotes such as yeast or in prokaryotes such as bacteria. Potentially suitable yeasts include Saccharomyces cerevisiae, Schizosaccharomyces pombe, Kluyvero-myces strains, Candida, or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments can be accomplished using known chemical or enzymatic methods. The polypeptide can also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). As used herein, an insect cell capable of expressing a nucleic acid of the present invention is “transformed.” Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”.

The polypeptide of the invention can be prepared by culturing transformed host cells under culture conditions suitable to express the recombinant polypeptide. The resulting expressed polypeptide can then be purified from such culture (i.e., from culture medium or cell extracts) using known purification processes, such as gel filtration and ion exchange chromatography. The purification of the polypeptide can also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the invention can also be expressed in a form which will facilitate purification. For example, it can be expressed as a fusion polypeptide, such as those of maltose binding polypeptide (MBP), glutathione-S-transferase (GST) or thioredoxin (TRX). Kits for expression and purification of such fusion polypeptides are commercially available from New England BioLabs (Beverly, Mass.), Pharmacia (Piscataway, N.J.) and InVitrogen, respectively. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to B7-H3A polypeptides, fragments, variants, binding partners etc. The polypeptide of the invention can also be expressed as a product of transgenic animals, e.g., as a component of the milk of transgenic cows, goats, pigs, or sheep which are characterized by somatic or germ cells containing a nucleotide sequence encoding the polypeptide.

It is also possible to utilize an affinity column comprising a polypeptide-binding polypeptide of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention. In this aspect of the invention, polypeptide-binding polypeptides, such as the anti-polypeptide antibodies of the invention or other polypeptides that can interact with the polypeptide of the invention, can be bound to a solid phase support such as a column chromatography matrix or a similar substrate suitable for identifying, separating, or purifying cells that express polypeptides of the invention on their surface. Adherence of polypeptide-binding polypeptides of the invention to a solid phase contacting surface can be accomplished by any means, for example, magnetic microspheres can be coated with these polypeptide-binding polypeptides and held in the incubation vessel through a magnetic field. Suspensions of cell mixtures are contacted with the solid phase that has such polypeptide-binding polypeptides thereon. Cells having polypeptides of the invention on their surface bind to the fixed polypeptide-binding polypeptide and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. Alternatively, mixtures of cells suspected of containing polypeptide-expressing cells of the invention first can be incubated with a biotinylated polypeptide-binding polypeptide of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The polypeptide can also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with polypeptides can possess biological properties in common therewith, including polypeptide activity. Thus, they can be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Agonists and Antagonists of B7-H3A Polypeptides

Any method which neutralizes B7-H3A polypeptides or inhibits expression of the B7-H3A genes (either transcription or translation) can be used to reduce the biological activities of B7-H3A polypeptides. In particular embodiments, antagonists inhibit the binding of at least one B7-H3A polypeptide to cells, thereby inhibiting biological activities induced by the binding of those B7-H3A polypeptides to the cells. In certain other embodiments of the invention, antagonists can be designed to reduce the level of endogenous B7-H3A gene expression, e.g., using well-known antisense or ribozyme approaches to inhibit or prevent translation of B7-H3A mRNA transcripts; triple helix approaches to inhibit transcription of B7-H3A family genes; or targeted homologous recombination to inactivate or “knock out” the B7-H3A genes or their endogenous promoters or enhancer elements. Such antisense, ribozyme, and triple helix antagonists can be designed to reduce or inhibit either unimpaired, or if appropriate, mutant B7-H3A gene activity. Techniques for the production and use of such molecules are well known to those of skill in the art.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a B7-H3A mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides that are complementary to the 5′ end of the message, e.g., the 5′ untranslated sequence up to and including the AUG initiation codon. However, oligonucleotides complementary to the 5′- or 3′-non-translated, non-coding regions of the B7-H3A gene transcript, or to the coding regions, could be used in an antisense approach to inhibit translation of endogenous B7-H3A mRNA. Oligonucleotides complementary to the 5′ untranslated region of the mRNA preferably include the complement of the AUG start codon. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci U.S.A. 86: 6553-6556; Lemaitre et al., 1987, Proc Natl Acad Sci 84: 648-652; PCT Publication No. WO88/09810), or hybridization-triggered cleavage agents or intercalating agents. (See, e.g., Zon, 1988, Pharm Res. 5: 539-549). The antisense molecules should be delivered to cells which express the B7-H3A transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous B7-H3A gene transcripts and thereby prevent translation of the B7-H3A mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave B7-H3A mRNA transcripts can also be used to prevent translation of B7-H3A mRNA and expression of B7-H3A polypeptides. (See, e.g., PCT International Publication WO90/11364, published Oct. 4, 1990; U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the B7-H3A polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous B7-H3A messages and inhibit translation. Because ribozymes, unlike antisense molecules, are catalytic, a lower intracellular concentration is required for efficiency.

Alternatively, endogenous B7-H3A gene expression can be reduced by targeting deoxyribonucleotide sequences complementary to the regulatory region of the target gene (i.e., the target gene promoter and/or enhancers) to form triple helical structures that prevent transcription of the target B7-H3A gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok A, Tabara H, and Mello CC, 2000, Genetic requirements for inheritance of RNAi in C. elegans, Science 287 (5462): 2494-2497), or the introduction of transgenes (Dernburg A F, Zalevsky J, Colaiacovo M P, and Villeneuve A M, 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are B7-H3A polypeptide variants with partner binding sites that have been altered in conformation so that (1) the B7-H3A variant will still bind to its partner(s), but a specified small molecule will fit into the altered binding site and block that interaction, or (2) the B7-H3A variant will no longer bind to its partner(s) unless a specified small molecule is present (see for example Bishop et al., 2000, Nature 407: 395-401). Nucleic acids encoding such altered B7-H3A polypeptides can be introduced into organisms according to methods described herein, and can replace the endogenous nucleic acid sequences encoding the corresponding B7-H3A polypeptide. Such methods allow for the interaction of a particular B7-H3A polypeptide with its binding partners to be regulated by administration of a small molecule compound to an organism, either systemically or in a localized manner.

The B7-H3A polypeptides themselves can also be employed in inhibiting a biological activity of B7-H3A in in vitro or in vivo procedures. Encompassed within the invention are extracellular domains of B7-H3A polypeptides, or fragments of such extracellular domains, that act as “dominant negative” inhibitors of native B7-H3A polypeptide function when expressed as fragments or as components of fusion polypeptides. For example, a purified polypeptide domain of the present invention, such as a domain comprising a combination of the V-like Ig domain and the C-like Ig domain, or either domain separately, can be used to inhibit binding of B7-H3A polypeptides to endogenous binding partners. Such use effectively would block B7-H3A polypeptide interactions and inhibit B7-H3A polypeptide activities. In still another aspect of the invention, a soluble form of the B7-H3A binding partner, which is expressed on T cells, is used to bind to and competitively inhibit activation of the endogenous B7-H3A polypeptide. Furthermore, antibodies which bind to B7-H3A polypeptides often inhibit B7-H3A polypeptide activity and act as antagonists. For example, antibodies that specifically recognize one or more epitopes of B7-H3A polypeptides, or epitopes of conserved variants of B7-H3A polypeptides, or peptide fragments of the B7-H3A polypeptide can be used in the invention to inhibit B7-H3A polypeptide activity. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified B7-H3A polypeptides of the present invention can be administered to modulate interactions between B7-H3A polypeptides and B7-H3A binding partners that are not membrane-bound. Such an approach will allow an alternative method for the modification of B7-H3A-influenced bioactivity.

In an alternative aspect, the invention further encompasses the use of agonists of B7-H3A polypeptide activity to treat or ameliorate the symptoms of a disease for which increased B7-H3A polypeptide activity is beneficial. Such diseases include but are not limited to cancer, including metastasis of cancer cells; bacterial or viral infections, including HIV infection; delayed reconstitution of T cells, for example following bone marrow transplantation; defects in T cell or accessory cell function, for example in hemodialysis patients subject to renal failure; and congenital immunodeficiencies. In a preferred aspect, the invention entails administering compositions comprising an B7-H3A nucleic acid or an B7-H3A polypeptide to cells in vitro, to cells ex vivo, to cells in vivo, and/or to a multicellular organism such as a vertebrate or mammal. Preferred therapeutic forms of B7-H3A are soluble forms, as described above. In still another aspect of the invention, the compositions comprise administering a B7-H3A-encoding nucleic acid for expression of a B7-H3A polypeptide in a host organism for treatment of disease. Particularly preferred in this regard is expression in a human patient for treatment of a dysfunction associated with aberrant (e.g., decreased) endogenous activity of a B7-H3A family polypeptide. Furthermore, the invention encompasses the administration to cells and/or organisms of compounds found to increase the endogenous activity of B7-H3A polypeptides. One example of compounds that increase B7-H3A polypeptide activity are agonistic antibodies, preferably monoclonal antibodies, that bind to B7-H3A polypeptides or binding partners, which may increase B7-H3A polypeptide activity by causing constitutive intracellular signaling (or “ligand mimicking”), or by preventing the binding of a native inhibitor of B7-H3A polypeptide activity.

Antibodies to B7-H3A Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with B7-H3A polypeptides, homologues, and variants, but not with other molecules. In one preferred embodiment, the antibodies are specific for the polypeptides of the present invention and do not cross-react with other polypeptides. In this manner, the B7-H3A polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hindrances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas. A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: a B7-H3A polypeptide, a fragment of a B7-H3A polypeptide, a functional equivalent of a B7-H3A polypeptide, or a mutant form of a B7-H3A polypeptide. Such host animals can include but are not limited to rabbits, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314:452-454; Morrison et al., 1984, Proc Natl Acad Sci USA 81:6851-6855; Boulianne et al., 1984, Nature 312:643646; Neuberger et al., 1985, Nature 314:268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat Biotechnol. 16(6): 535-539; and U.S. Pat. No. 5,969,108).

Antigen-binding antibody fragments which recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against B7-H3A gene products. Single chain antibodies are formed by linking the heavy and light chain fragments of the Fv region via an amino acid bridge, resulting in a single chain polypeptide. Such single chain antibodies can also be useful intracellularly (i.e., as ‘intrabodies), for example as described by Marasco et al. (J. Immunol. Methods 231:223-238, 1999) for genetic therapy in HIV infection. In addition, antibodies to the B7-H3A polypeptide can, in turn, be utilized to generate anti-idiotype antibodies that “mimic” the B7-H3A polypeptide and that may bind to the B7-H3A polypeptide's binding partners using techniques well known to those skilled in the art. (See, e.g., Greenspan & Bona, 1993, FASEB J 7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using. the leucine zipper moieties from the Fos and Jun proteins (which preferentially form heterodimers) as described by Kostelny et al. (J. Immunol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface B7-H3A, induce biological effects (e.g., transduction of biological signals) similar to the biological effects induced when the B7-H3A binding partner binds to cell surface B7-H3A. Agonistic antibodies can be used to induce B7-H3A-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety). Bispecific antibodies that bind B7-H3A polypeptides of the invention via a first antigen binding domain will be useful in diagnostic applications and in treating immunological and/or T cell costimulation-related conditions. Examples of polypeptides (or other antigens) that the inventive bispecific antibodies can bind via a second antigen binding domain include other B7 polypeptides such as B7-H3, and T cell receptors such as ICOS, PD-1, and similar Ig superfamily molecules.

Those antibodies that can block binding of the B7-H3A polypeptides of the invention to binding partners for B7-H3A can be used to inhibit B7-H3A-mediated intercellular communication or cell stimulation that results from such binding. Such blocking antibodies can be identified using any suitable assay procedure, such as by testing antibodies for the ability to inhibit binding of B7-H3A binding to certain cells expressing an B7-H3A binding partner. Alternatively, blocking antibodies can be identified in assays for the ability to inhibit a biological effect that results from binding of soluble B7-H3A to target cells. Antibodies can be assayed for the ability to inhibit B7-H3A binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of B7-H3A with cell surface binding partner receptor thus can be treated. A therapeutic method involves in vivo administration of a blocking antibody to a mammal in an amount effective in inhibiting B7-H3A binding partner-mediated biological activity. Monoclonal antibodies are generally preferred for use in such therapeutic methods. In one embodiment, an antigen-binding antibody fragment is employed. Compositions comprising an antibody that is directed against B7-H3A, and a physiologically acceptable diluent, excipient, or carrier, are provided herein. Suitable components of such compositions are as described below for compositions containing B7-H3A polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the invention by immunoaffinity chromatography.

Rational Design of Compounds that Interact with B7-H3A Polypeptides

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the polypeptide or which enhance or interfere with the function of a polypeptide in vivo (Hodgson J (1991) Biotechnology 9:19-21). In one approach, the three-dimensional structure of a polypeptide of interest, or of a polypeptide-inhibitor complex, is determined by x-ray crystallography, by nuclear magnetic resonance, or by computer homology modeling or, most typically, by a combination of these approaches. Both the shape and charges of the polypeptide must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous B7-H3A-like molecules, to identify efficient inhibitors, or to identify small molecules that bind B7-H3A polypeptides. Useful examples of rational drug design can include molecules which have improved activity or stability as shown by Braxton S and Wells J A (1992 Biochemistry 31:7796-7801) or which act as inhibitors, agonists, or antagonists of native peptides as shown by Athauda S B et al (1993 J Biochem 113:742-746). The use of B7-H3A polypeptide structural information in molecular modeling software systems to assist in inhibitor design and inhibitor-B7-H3A polypeptide interaction is also encompassed by the invention. A particular method of the invention comprises analyzing the three dimensional structure of B7-H3A polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

Assays of B7-H3A Polypeptide Activities

The purified B7-H3A polypeptides of the invention (including polypeptides, polypeptides, fragments, variants, oligomers, and other forms) are useful in a variety of assays. For example, the B7-H3A molecules of the present invention can be used to identify binding partners of B7-H3A poly-peptides, which can also be used to modulate intercellular communication, cell stimulation, or immune cell activity. Alternatively, they can be used to identify non-binding-partner molecules or substances that modulate intercellular communication, cell stimulatory pathways, or immune cell activity.

Assays to Identify Binding Partners. Polypeptides of the B7-H3A family and fragments thereof can be used to identify binding partners. For example, they can be tested for the ability to bind a candidate binding partner in any suitable assay, such as a conventional binding assay. To illustrate, the B7-H3A polypeptide can be labeled with a detectable reagent (e.g., a radionuclide, chromophore, enzyme that catalyzes a colorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al., (EMBO J. 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82: 8672-8676), homogeneous time-resolved fluorescence methods (Park et al., 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden). Compounds that can be assayed for binding to B7-H3A polypeptides include but are not limited to small organic molecules, such as those that are comerically available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co.(Trevillett, Cornwall, UK), MDS Panlabs (Bothell, Wash.), Pharmacopeia (Princeton, N.J.), and Trega (San Diego, Calif.). Preferred small organic molecules for screening using these assays are usually less than 10K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2): 193-198). Compounds including natural products, inorganic chemicals, and biologically active materials such as proteins and toxins can also be assayed using these methods for the ability to bind to B7-H3A polypeptides.

Yeast Two-Hybrid or “Interaction Trap” Assays. Where the B7-H3A polypeptide binds or potentially binds to another polypeptide (such as, for example, in a receptor-ligand interaction), the nucleic acid encoding the B7-H3A polypeptide can also be used in interaction trap assays (such as, for example, that described in Gyuris et al., Cell 75:791-803 (1993)) to identify nucleic acids encoding the other polypeptide with which binding occurs or to identify inhibitors of the binding interaction. Polypeptides involved in these binding interactions can also be used to screen for peptide or small molecule inhibitors or agonists of the binding interaction.

Competitive Binding Assays. Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled B7-H3A and intact cells expressing B7-H3A (endogenous or recombinant) on the cell surface. For example, a radiolabeled soluble B7-H3A fragment can be used to compete with a soluble B7-H3A variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Assays to Identify Modulators of Intercellular Communication, Cell Stimulation, or Immune Cell Activity. The influence of B7-H3A on intercellular communication, cell stimulation, or immune cell activity can be manipulated to control these activities in target cells. For example, the disclosed B7-H3A polypeptides, nucleic acids encoding the disclosed B7-H3A polypeptides, or agonists or antagonists of such polypeptides can be administered to a cell or group of cells to induce, enhance, suppress, or arrest cellular communication, cell stimulation, or activity in the target cells. Identification of B7-H3A polypeptides, agonists or antagonists that can be used in this manner can be carried out via a variety of assays known to those skilled in the art. Included in such assays are those that evaluate the ability of an B7-H3A polypeptide to influence intercellular communication, cell stimulation or activity. Such an assay would involve, for example, the analysis of immune cell interaction in the presence of an B7-H3A polypeptide. In such an assay, one would determine a rate of communication or cell stimulation in the presence of the B7-H3A polypeptide and then determine if such communication or cell stimulation is altered in the presence of a candidate agonist or antagonist or another B7-H3A polypeptide. Exemplary assays for this aspect of the invention include cytokine secretion assays, T-cell co-stimulation assays, and mixed lymphocyte reactions involving antigen presenting cells and T cells. These assays are well known to those skilled in the art.

In another aspect, the present invention provides a method of detecting the ability of a test compound to affect the intercellular communication or cell stimulatory activity of a cell. In this aspect, the method comprises: (1) contacting a first group of target cells with a test compound including an B7-H3A receptor polypeptide or fragment thereof under conditions appropriate to the particular assay being used; (2) measuring the net rate of intercellular communication or cell stimulation among the target cells; and (3) observing the net rate of intercellular communication or cell stimulation among control cells containing the B7-H3A receptor polypeptides or fragments thereof, in the absence of a test compound, under otherwise identical conditions as the first group of cells. In this embodiment, the net rate of intercellular communication or cell stimulation in the control cells is compared to that of the cells treated with both the B7-H3A molecule as well as a test compound. The comparison will provide a difference in the net rate of intercellular communication or cell stimulation such that an effector of intercellular communication or cell stimulation can be identified. The test compound can function as an effector by either activating or up-regulating, or by inhibiting or down-regulating intercellular communication or cell stimulation, and can be detected through this method.

Cell Proliferation, Cell Death, Cell Differentiation, and Cell Adhesion Assays. A polypeptide of the present invention may exhibit cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of a polypeptide of the present invention is evidenced by any one of a number of routine factor-dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK. The activity of a B7-H3A polypeptide of the invention may, among other means, be measured by the following methods:

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7: Immunologic studies in humans); Takai et al., J. Immunol. 137: 3494-3500, 1986; Bertagnolli et al., J. Immunol. 145: 1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173: 1205-1211, 1991; Moreau et al., Nature 336:690-692, 1988; Greenberger et al., Proc Natl Acad. Sci. USA 80: 2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83: 1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., Proc Natl Acad Sci USA 77: 6091-6095, 1980; Weinberger et al., Eur. J. Immun. 11:405-411, 1981; Takai et al., J. Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144: 3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., Proc Natl Acad. Sci. USA 88:7548-7551, 1991

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, polypeptides that regulate lympho-hematopoiesis) include, without limitation, those described in: Methylcellulose colony forming assays, Freshney, 1994, In Culture of Hematopoietic Cells, Freshney et al. eds. pp. 265-268, Wiley-Liss, Inc., New York, N.Y.; Hirayama et al., Proc. Natl. Acad. Sci. USA 89:5907-5911, 1992; Primitive hematopoietic colony forming cells with high proliferative potential, McNiece and Briddell, 1994, In Culture of Hematopoietic Cells, Freshney et al. eds. pp. 23-39, Wiley-Liss, Inc., New York, N.Y.; Neben et al., Experimental Hematology 22:353-359, 1994; Ploemacher, 1994, Cobblestone area forming cell assay, In Culture of Hematopoietic Cells, Freshney et al. eds. pp. 1-21, Wiley-Liss, Inc., New York, N.Y.; Spooncer et al., 1994, Long term bone marrow cultures in the presence of stromal cells, In Culture of Hematopoietic Cells, Freshney et al. eds. pp. 163-179, Wiley-Liss, Inc., New York, N.Y.; Sutherland, 1994, Long term culture initiating cell assay, In Culture of Hematopoietic Cells, Freshney et al. eds. Vol pp. 139-162, Wiley-Liss, Inc., New York, N.Y.

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Diagnostic and Other Uses of B7-H3A Polypeptides and Nucleic Acids

The nucleic acids encoding the B7-H3A polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and. for gene therapy. Uses of B7-H3A polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987

Probes and Primers. Among the uses of the disclosed B7-H3A nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human B7-H3A homologues.

Diagnostics and Gene Therapy. The nucleic acids encoding B7-H3A polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The B7-H3A polypeptides of the invention each can be used as reagents in methods to screen for or identify binding partners. For example, the B7-H3A polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to protein A- or protein G-containing chromatography columns through interaction with the Fc moiety. The B7-H3A polypeptides also find use in identifying cells that express a binding partner on the cell surface. Polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing potential binding-partner-expressing cells are contacted with the solid phase having the polypeptides thereon. Cells expressing the binding partner on the cell surface bind to the fixed polypeptides, and unbound cells are washed away. Alternatively, B7-H3A polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (see Berenson, et al. J. Cell. Biochem., 10D:239, 1986). Washing to remove unbound material, and the release of the bound cells, are performed using conventional methods. In some instances, the above methods for screening for or identifying binding partners may also be used or modified to isolate or purify such binding partner molecules or cells expressing them.

Measuring Biological Activity. Polypeptides also find use in measuring the biological activity of B7-H3A-binding polypeptides in terms of their binding affinity. The polypeptides thus can be employed by those conducting “quality assurance” studies, e.g., to monitor shelf life and stability of polypeptide under different conditions. For example, the polypeptides can be employed in a binding affinity study to measure the biological activity of a binding partner polypeptide that has been stored at different temperatures, or produced in different cell types. The polypeptides also can be used to determine whether biological activity is retained after modification of a binding partner polypeptide (e.g., chemical modification, truncation, mutation, etc.). The binding affinity of the modified polypeptide is compared to that of an unmodified binding polypeptide to detect any adverse impact of the modifications on biological activity of the binding polypeptide. The biological activity of a binding polypeptide thus can be ascertained before it is used in a research study, for example.

Carriers and Delivery Agents. The polypeptides also find use as carriers for delivering agents attached thereto to cells bearing identified binding partners. The polypeptides thus can be used to deliver diagnostic or therapeutic agents to such cells (or to other cell types found to express binding partners on the cell surface) in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent, for example. Conjugates comprising polypeptides and a suitable diagnostic or therapeutic agent (preferably covalently linked) are thus prepared. The conjugates are administered or otherwise employed in an amount appropriate for the particular application.

Treating Diseases with B7-H3A Polypeptides and Antagonists Thereof

It is anticipated that the B7-H3A polypeptides, fragments, variants, antagonists, agonists, antibodies, and binding partners of the invention will be useful for treating medical conditions and diseases including, but not limited to, immunological conditions as described further herein. The therapeutic molecule or molecules to be used will depend on the etiology of the condition to be treated and the biological pathways involved, and variants, fragments, and binding partners of B7-H3A polypeptides may have effects similar to or different from B7-H3A polypeptides. For example, an antagonist of the immunogenic activity of B7-H3A polypeptides can be selected for treatment of conditions involving excess T cell activity, but a particular fragment of a given B7-H3A polypeptide may also act as an effective dominant negative antagonist of that activity. Therefore, in the following paragraphs “B7-H3A polypeptides or agonists thereof” refers to all B7-H3A polypeptides, fragments, variants, agonists, antibodies, and binding partners etc. of the invention that increase, enhance, or promote B7-H3A polypeptide activity. “Antagonists of B7-H3A polypeptide activity” refers to all B7-H3A polypeptide fragments, variants, antagonists, antibodies, and binding partners etc. of the invention that decrease, inhibit, suppress, or eliminate B7-H3A polypeptide activity. “B7-H3A polypeptides or antagonists” refers to all B7-H3A polypeptides, fragments, variants, antagonists, agonists, antibodies, and binding partners etc. of the invention, and it is understood that a specific molecule or molecules can be selected from those provided as embodiments of the invention by individuals of skill in the art, according to the biological and therapeutic considerations described herein.

The disclosed B7-H3A polypeptides or agonists thereof, compositions and combination therapies described herein are useful in medicines for treating bacterial, viral or protozoal infections, and complications resulting therefrom. One such disease is Mycoplasma pneumonia. In addition, provided herein is the use of B7-H3A polypeptides or agonists thereof to treat AIDS and related conditions, such as AIDS dementia complex, AIDS associated wasting, lipidistrophy due to antiretroviral therapy; and Kaposi's sarcoma. Provided herein is the use of B7-H3A polypeptides or agonists thereof for treating protozoal diseases, including malaria and schistosomiasis. Additionally provided is the use of B7-H3A polypeptides or agonists thereof to treat erythema nodosum leprosum; bacterial or viral meningitis; tuberculosis, including pulmonary tuberculosis; and pneumonitis secondary to a bacterial or viral infection. Provided also herein is the use of B7-H3A polypeptides or agonists thereof to prepare medicaments for treating louse-borne relapsing fevers, such as that caused by Borrelia recurrentis. The B7-H3A polypeptides of the invention or agonists thereof can also be used to prepare a medicament for treating conditions caused by Herpes viruses, such as herpetic stromal keratitis, corneal lesions, and virus-induced corneal disorders. In addition, B7-H3A polypeptides or agonists thereof can be used in treating human papillomavirus infections. The B7-H3A polypeptides of the invention or agonists thereof are used also to prepare medicaments to treat influenza.

Cardiovascular disorders resulting from inflammation or autoimmune conditions are treatable with antagonists of B7-H3A polypeptide activity, pharmaceutical compositions or combination therapies, including complications of coronary by-pass surgery; ischemia/reperfusion injury; heart disease, including chronic autoimmune myocarditis and viral myocarditis; heart failure, including chronic heart failure (CHF), cachexia of heart failure; restenosis after heart surgery; and post-implantation complications of left ventricular assist devices.

Provided also are methods for using antagonists of B7-H3A polypeptide activity, compositions or combination therapies to treat various inflammatory and/or autoimmune disorders including autoimmune diabetes; Hashimoto's thyroiditis (i.e., autoimmune thyroiditis); autoimmune hemolytic anemia; autoimmune disorders or diseases associated with hereditary deficiencies; demyelinating neuropathy; inflammation of the liver due to unknown causes; the inflammatory response occuring prior, during, or after the transfusion of allogeneic red blood cells in cardiac or other surgery, or in treating a traumatic injury to a limb or joint, such as traumatic knee injury; inflammatory eye disease, including inflammatory eye disease associated with smoking; and inner ear or cochlear nerve-associated hearing loss that is thought to result from an autoimmune process, i.e., autoimmune hearing loss. This autoimmune hearing loss condition currently is treated with steroids, methotrexate and/or cyclophosphamide, which may be administered concurrently with antagonists of B7-H3A polypeptide activity.

Inflammatory and/or autoimmune conditions of the gastrointestinal system or genitourinary system also are treatable with antagonists of B7-H3A polypeptide activity, compositions or combination therapies, such as Crohn's disease; ulcerative colitis; and autoimmune glomerulonephritis.

Also provided herein are methods for using B7-H3A polypeptides or agonists thereof, compositions or combination therapies to treat various oncologic disorders. For example, B7-H3A polypeptides or agonists thereof are used to treat various forms of cancer, including acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. Additional diseases treatable with the subject B7-H3A polypeptides or agonists thereof, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating leukemia, including acute myelogenous leukemia, chronic or acute lymphoblastic leukemia and hairy cell leukemia. Other malignancies with invasive metastatic potential can be treated with the subject compounds, compositions and combination therapies, including multiple myeloma. Various lymphoproliferative disorders also are treatable with the disclosed B7-H3A polypeptides or agonists thereof, compositions or combination therapies. These include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

A number of pulmonary disorders also can be treated with antagonists of B7-H3A polypeptide activity, compositions and combination therapies, such as allergies, including allergic rhinitis, contact dermatitis, atopic dermatitis, and asthma.

Other embodiments provide methods for using antagonists of B7-H3A polypeptide activity, compositions or combination therapies to treat a variety of rheumatic disorders. These include: adult and juvenile rheumatoid arthritis; systemic lupus erythematosus; gout; osteoarthritis; polymyalgia rheumatica; seronegative spondylarthropathies, including ankylosing spondylitis; and Reiter's disease. The subject antagonists of B7-H3A polypeptide activity, compositions and combination therapies are used also to treat psoriatic arthritis and chronic Lyme arthritis. Also treatable with these compounds, compositions and combination therapies are Still's disease and uveitis associated with rheumatoid arthritis. In addition, the compounds, compositions and combination therapies of the invention are used in treating disorders resulting in inflammation of the voluntary muscle, including dermatomyositis and polymyositis. Moreover, the compounds, compositions ant combinations disclosed herein are useful for treating sporadic inclusion body myositis, as TNFα may play a significant role in the progression of this muscle disease. In addition, the compounds, compositions and combinations disclosed herein are used to treat multicentric reticulohistiocytosis, a disease in which joint destruction and papular nodules of the face and hands are associated with excess production of proinflammatory cytokines by multinucleated giant cells.

Cervicogenic headache is a common form of headache arising from dysfunction in the neck area, and which is associated with elevated levels of TNFα, which are believed to mediate an inflammatory condition that contributes to the patient's discomfort (Martelletti, Clin Exp Rheumatol 18(2 Suppl 19):S33-8 (Mar-Apr, 2000)). Cervicogenic headache may be treated by administering antagonists of B7-H3A polypeptide activity as disclosed herein, thereby reducing the inflammatory response and associated headache pain.

Disorders associated with transplantation also are treatable with antagonists of B7-H3A polypeptide activity, compositions or combination therapies, such as graft-versus-host disease, and complications resulting from solid organ transplantation, including transplantion of heart, liver, lung, skin, kidney or other organs. Antagonists of B7-H3A polypeptide activity may be administered, for example, to prevent or inhibit the development of bronchiolitis obliterans after lung transplantation.

Disorders involving the skin or mucous membranes also are treatable using antagonists of B7-H3A polypeptide activity, compositions or combination therapies. Such disorders include inflammatory skin disease and psoriasis.

Administration of B7-H3A Polypeptides and Antagonists Thereof

This invention provides compounds, compositions, and methods for treating a patient, preferably a mammalian patient, and most preferably a human patient, who is suffering from a medical disorder, and in particular a B7-H3A-mediated disorder. Such B7-H3A-mediated disorders include conditions caused (directly or indirectly) or exacerbated by binding between B7-H3A and a binding partner. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g., prophylactic) and palliative or ameliorative treatment. For such therapeutic uses, B7-H3A polypeptides and fragments, B7-H3A nucleic acids encoding the B7-H3A family polypeptides, and/or agonists or antagonists of the B7-H3A polypeptide such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble B7-H3A polypeptides.

Therapeutically Effective Amount. In practicing the method of treatment or use of the present invention, a therapeutically effective amount of a therapeutic agent of the present invention is administered to a patient having a condition to be treated, preferably to treat or ameliorate diseases associated with the activity of a B7-H3A family polypeptide. “Therapeutic agent” includes without limitation any of the B7-H3A polypeptides, fragments, and variants; nucleic acids encoding the B7-H3A family polypeptides, fragments, and variants; agonists or antagonists of the B7-H3A polypeptides such as antibodies; B7-H3A polypeptide binding partners; complexes formed from the B7-H3A family polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as B7-H3A polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose may be sufficient. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, B7-H3A polypeptides or antagonists are administered one time per week to treat the various medical disorders disclosed herein, in another embodiment is administered at least two times per week, and in another embodiment is administered at least three times per week. If injected, the effective amount of B7-H3A polypeptides or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing B7-H3A polypeptides or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of B7-H3A polypeptides or antagonists one to three times per week over a period of at least three weeks, or a dose of 50 mg of B7-H3A polypeptides or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of B7-H3A polypeptides or antagonists, administered by subcutaneous injection one or more times per week. If an antibody against a B7-H3A polypeptide is used as the B7-H3A polypeptide antagonist, a preferred dose range is 0.1 to 20 mg/kg, and more preferably is 1-10 mg/kg. Another preferred dose range for an anti-B7-H3A polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of a B7-H3A polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of B7-H3A polypeptides are used. Sustained-release forms suitable for use in the disclosed methods include, but are not limited to, B7-H3A polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. A B7-H3A polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of B7-H3A polypeptides or antagonists concurrently with one or more other drugs that are administered to the same patient in combination with the B7-H3A polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be included in the pharmaceutical composition of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15, IL-17, IL-18, IFN, TNF0, TNF1, TNF2, G-CSF, Meg-CSF, thrombopoietin, stem cell factor, and erythropoietin. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a B7-H3A polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, B7-H3A polypeptides or antagonists can be combined with a second B7-H3A polypeptide/antagonist, including an antibody against a B7-H3A polypeptide, or a B7-H3A polypeptide-derived peptide that acts as a competitive inhibitor of a native B7-H3A polypeptide.

Routes of Administration. Any efficacious route of administration can be used to therapeutically administer B7-H3A polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation.; eyedrops; vaginal or rectal suppositories; buccal preparations; oral preparations, including pills, syrups, lozenges or chewing gum; and topical preparations such as lotions, gels, sprays, ointments or other suitable techniques. Alternatively, polypeptideaceous B7-H3A polypeptides or antagonists may be administered by implanting cultured cells that express the polypeptide, for example, by implanting cells that express B7-H3A polypeptides or antagonists. Cells may also be cultured ex vivo in the presence of polypeptides of the present invention in order to proliferate or to produce a desired effect on or activity in such cells. Treated cells can then be introduced in vivo for therapeutic purposes. In another embodiment, the patient's own cells are induced to produce B7-H3A polypeptides or antagonists by transfection in vivo or ex vivo with a DNA that encodes B7-H3A polypeptides or antagonists. This DNA can be introduced into the patient's cells, for example, by injecting naked DNA or liposome-encapsulated DNA that encodes B7-H3A polypeptides or antagonists, or by other means of transfection. Nucleic acids of the invention can also be administered to patients by other known methods for introduction of nucleic acid into a cell or organism (including, without limitation, in the form of viral vectors or naked DNA). When B7-H3A polypeptides or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a polypeptide of the invention which can also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, B7-H3A polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a TNFα-mediated inflammatory or arthritic condition. In such instances, an appropriate dose can be determined according to the animal's body weight. For example, a dose of 0.2-1 mg/kg may be used. Alternatively, the dose is determined according to the animal's surface area, an exemplary dose ranging from 0.1-20 mg/M², or more preferably, from 5-12 mg/m². For small animals, such as dogs or cats, a suitable dose is 0.4 mg/kg. In a preferred embodiment, B7-H3A polypeptides or antagonists (preferably constructed from genes derived from the same species as the patient), is administered by injection or other suitable route one or more times per week until the animal's condition is improved, or it can be administered indefinitely.

Manufacture of Medicaments. The present invention also relates to the use B7-H3A polypeptides, fragments, and variants; nucleic acids encoding the B7-H3A family polypeptides, fragments, and variants; agonists or antagonists of the B7-H3A polypeptides such as antibodies; B7-H3A polypeptide binding partners; complexes formed from the B7-H3A family polypeptides, fragments, variants, and binding partners, etc, in the manufacture of a medicament for the prevention or therapeutic treatment of each medical disorder disclosed herein.

Use of B7-H3A Polypeptides and Antagonists Thereof as Adjuvants

An effective vaccine must induce an appropriate immune response to the correct antigen or antigens. The immune system uses many mechanisms for attacking pathogens, but not all of these are activated after immunization. Protective immunity induced by vaccination is dependent on the capacity of the vaccine to elicit the appropriate immune response to resist, control, or eliminate the pathogen. Depending on the pathogen, this may require a humoral immune response, which involves antibodies and other factors such as complement, and/or a cell-mediated immune response, which is mediated by cells such as cytotoxic T cells. The type of immune response that is produced is determined by the nature of the T cells that develop after immunization. For example, many bacterial, protozoal, and intracellular parasitic and viral infections appear to require a strong cell-mediated immune response for protection, while other pathogens such as helminths primarily respond to a humoral response. The current paradigm of the role of T cells in the particular immune response is that CD4⁺ T cells can be separated into subsets on the basis of the repertoire of cytokines produced and that the distinct cytokine profile observed in these cells determines their function. This T cell model includes two major subsets: Th1 cells that produce IL-2 and interferon gamma (IFN-gamma) and mediate cellular immune responses, and Th2 cells that produce IL-4, IL-5, and IL-10 and augment humoral immune responses (Mosmann et al., 1986, J Immunol 126: 2348).

Many vaccine compositions employ adjuvants, that is, substances which enhance the immune response when administered together with an immunogen or antigen. Adjuvants are thought to function in one or more of several possible ways, including increasing the surface area of antigen; prolonging the retention of the antigen in the body thus allowing time for the lymphoid system to have access to the antigen; slowing the release of antigen; targeting antigen to macrophages; increasing antigen uptake; up-regulating antigen processing; stimulating cytokine release; stimulating B cell switching and maturation and/or eliminating immuno-suppressor cells; activating macrophages, dendritic cells, B cells and T cells; or otherwise eliciting non-specific activation of the cells of the immune system (see, for example, Warren et al., 1986, Annu Rev Immunol 4: 369). Many of the most effective adjuvants include bacteria or their products, e.g., microorganisms such as the attenuated strain of Mycobacterium bovis, bacillus Calmette-Guerin (BCG); microorganism components, e.g., alum-precipitated diphtheria toxoid, bacterial lipopolysaccharide and endotoxins. Despite their immuno-stimulating properties, many bacterial adjuvants have toxic or other negative effects, particularly in humans. For example, such a large population has been exposed to some of the bacterial adjuvants, like BCG, that there is a danger of eliciting a secondary response with future use as a vaccine adjuvant. Heat-killed bacteria, being non-native to mammalian hosts, also risk causing toxic effects in the host. Alternative adjuvants that stimulate or enhance the host's immune responses without inducing a toxic effect, and which are suitable for use in pharmaceutical compositions, such as vaccines, are particularly useful. Also, an essential role of adjuvants in vaccines is to modulate CD4⁺ T cell subset differentiation. The ability of an adjuvant to induce and increase a specific type of effector T cell (Th1 or Th2) and thus a specific type of immune response (cell-mediated or humoral) is a key factor in the selection of particular adjuvants for vaccine use against a particular pathogen. The present invention provides the use of B7-H3A polypeptides and agonists thereof as adjuvants in vaccines, in order to promote the production of Th1 cells by the vaccine, and/or to increase the immunogenicity of the vaccine, which is useful for example when the vaccine is meant to increase immune activity toward an antigen. Also provided by the present invention is the use of antagonists of B7-H3A polypeptide activity as adjuvants in vaccines, in order to promote the production of Th2 cells by the vaccine, and/or to increase immunotolerance (for example, in order to decrease autoimmunity or reaction to allergenic antigens) or to modify the immune response produced by the vaccine.

Antigens are substances which are capable, under appropriate conditions, of inducing a specific immune response and of reacting with the products of that response, such as specific antibodies or T cells, or both. A vaccine is a composition comprising antigenic moieties, usually consisting of inactivated infectious agents or of allergens, or, some part of an infectious agent or allergen, that is injected into the body to produce active immunity, or in the case of allergens, to induce tolerance. Antigens that can be used in the present invention are compounds which, when introduced into a mammal, preferably a human, will result in the formation of antibodies and/or cell-mediated immunity. Representative of the antigens that can be used according to the present invention include, but are not limited to live or killed viruses and other microorganisms; natural, recombinant or synthetic products derived from viruses, bacteria, fungi, parasites and other infectious agents; antigens promoting autoimmune diseases, hormones, or tumor antigens which might be used in prophylactic or therapeutic vaccines; and allergens (see the Table below). The viral or microorganismal products can be components which the organism produced by enzymatic cleavage or can be components of the organism (proteins, polypeptides, polysaccharides, nucleic acids, lipids, etc.) that were produced by recombinant DNA techniques that are well-known to those of ordinary skill in the art. The antigen component of the vaccine may also comprise one or several antigenic molecules such as haptens, which are small antigenic determinants capable of eliciting an immune response only when coupled to a carrier.

| Antigen | |
|---|---|
| Category | Some Specific Examples of Representative Antigens |
| Viruses | Rotavirus; foot and mouth disease; influenza, |
| | including influenza A and B; parainfluenza; |
| | Herpes species (Herpes simplex, Epstein-Barr |
| | virus, chicken pox, pseudorabies, |
| | cytomegalovirus); rabies; polio; hepatitis A; |
| | hepatitis B; hepatitis C; hepatitis E; measles; |
| | distemper; Venezuelan equine encephalomyelitis; |
| | feline leukemia virus; reovirus; respiratory |
| | syncytial virus; bovine respiratory syncytial |
| | virus; Lassa fever virus; polyoma tumor virus; |
| | parvovirus; canine parvovirus; papilloma virus; |
| | tick-borne encephalitis; rinderpest; human |
| | rhinovirus species; enterovirus species; Mengo |
| | virus; paramyxovirus; avian infectious bronchitis |
| | virus; HTLV 1; HIV-1; HIV-2; LCMV (lymphocytic |
| | choriomeningitis virus); adenovirus; togavirus |
| | (rubella, yellow fever, dengue fever); coronavirus |
| Bacteria | Bordetella pertussis; Brucella abortis; |
| | Escherichia coli; Salmonella species including |
| | Salmonella typhi; streptococci; Vibrio species |
| | (V. cholera, V. parahaemolyticus); Shigella |
| | species; Pseudomonas species; Brucella species; |
| | Mycobacteria species (tuberculosis, avium, BCG, |
| | leprosy); pneumococci; staphlylococci; |
| | Enterobacter species; Rochalimaia henselae; |
| | Pasterurella species (P. haemolytica, P. |
| | multocida); Chlamydia species (C. trachomatis, |
| | C. psittaci, Lymphogranuloma venereum); Syphilis |
| | (Treponema pallidum); Haemophilus species; |
| | Mycoplasma species; Lyme disease (Borrelia |
| | burgdorferi); Legionnaires' disease; Botulism |
| | (Colstridium botulinum); Corynebacterium |
| | diphtheriae; Yersinia entercolitica |
| Ricketsial | Rocky mountain spotted fever; thyphus; |
| Infections | Ehrlichia species |
| Parasites | Malaria (Plasmodium falciparum, P. vivax, P. |
| and | malariae); schistosomes; trypanosomes; Leishmania |
| Protozoa | species; filarial nematodes; trichomoniasis; |
| | sarcosporidiasis; Taenia species (T. saginata, T. |
| | solium); Toxoplasma gondii; trichinelosis |
| | (Trichinella spiralis); coccidiosis |
| | (Eimeria species) |
| Fungi | Cryptococcus neoformans; Candida albicans; |
| | Apergillus fumigatus; coccidioidomycosis |
| Recombinant | Herpes simplex; Epstein-Barr virus; hepatitis B; |
| Proteins | pseudorabies; flavivirus (dengue, yellow fever); |
| | Neisseria gonorrhoeae; malaria: circumsporozoite |
| | protein, merozoite protein; trypanosome surface |
| | antigen protein; pertussis; alphaviruses; |
| | adenovirus |
| Proteins | Diphtheria toxoid; tetanus toxoid; meningococcal |
| | outer membrane protein (OMP); |
| | streptococcal M protein; hepatitis B; influenza |
| | hemagglutinin; cancer antigen; tumor antigens; |
| | toxins; exotoxins; neurotoxins; cytokines and |
| | cytokine receptors; monokines and monokine |
| | receptors |
| Synthetic | Malaria; influenza; foot and mouth disease virus; |
| Peptides | hepatitis B; hepatitis C |
| Poly- | Pneumococcal polysaccharide; Haemophilis influenza |
| saccharides | polyribosyl-ribitolphosphate (PRP); Neisseria |
| | meningitides; Pseudomonas aeruginosa; Klebsiella |
| | pneumoniae |
| Oligo- | Pneumococcal |
| saccharide | |
| Allergens | Plant pollens; animal dander; dust mites, Blatella |
| | species antigens (Bla g 1, 2, or 5), Periplaneta |
| | species antigens (Per a 1) |

Adjuvants are compounds that, when used in combination with specific vaccine antigens, augment or otherwise alter or modify the resultant immune responses. Modification of the immune response means augmenting, intensifying, or broadening the specificity of either or both antibody and cellular immune responses. Modification of the immune response can also mean decreasing or suppressing certain antigen-specific immune responses, for example, in the induction of tolerance toward an allergen. Modification of the immune response by the adjuvant may increase the overall titer of antibodies specific for the vaccine antigen and/or induce cellular immune responses specific for the vaccine antigen, so that effective vaccination can be made using lower amounts of antigen. Methods for detecting modification of the immune response by the adjuvant include several well-known assays such as ELISA (enzyme-linked immunosorbent assay), which measures the titer of antigen-specific antibodies, and the ELISPOT (enzyme-linked immunospot) assay, which allows ex vivo quantification of antigen-reactive T cells and of cells producing antigen-specific antibodies (see, for example, Zigterman et al., 1988, J Immunol Methods 106: 101-107; U.S. Pat. No. 6,149,922; and U.S. Pat. No. 6,153,182). Variations of ELISA in which biotin/avidin interactions are used to create antibody-antigen-antibody ‘bridges’ or ‘sandwiches’ are also well known in the art (see, for example, U.S. Pat. No. 6,149,922). In order to measure the effect of an adjuvant preparation on the production of functional, neutralizing antibodies, influenza virus hemagglutinin (HA) can be used as an antigen, animals are immunized with HA with differing amounts of adjuvant, and the ability of the resulting serum antibodies to inhibit the hemagglutinin-dependent agglutination of red blood cells can be determined using a hemagglutination inhibition (HAI) assay, essentially as described by the CDC Manual (U.S. Department of Health and Human Services/Public Health Service/Centers for Disease Control, 1982, Concepts and Procedures for Laboratory Based Influenza Surveillance) and U.S. Pat. No. 6,149,922. These assays allow the effects of supplementing a vaccine with B7-H3A polypeptides or antagonists to be investigated by determining antibody titers and the kinetics of antibody responses. For example, dose-titration studies of a vaccine can be done to identify doses that induce measurable antibody responses after a single immunization. Antibody responses are followed for 30, 60, or 90 or more days and dose levels that are optimally and suboptimally immunogenic can be identified. Also, vaccine formulations containing these dose levels and supplemented with increasing amounts of adjuvant (B7-H3A polypeptide or antagonist) can be evaluated and active doses of adjuvant identified. The kinetics and duration of antibody responses can evaluated by extension of the observation and antibody testing period to 6 months or more (see, for example, U.S. Pat. No. 6,149,922). Modulation of the immune response by adjuvant can also be assessed by measuring the antigen-dependent proliferation of T cells from immunized mice in a ³H-thymidine uptake assay (see, for example, U.S. Pat. No. 6,051,227 and U.S. Pat. No. 6,153,182). Other T cell responses to immunization with varying amounts of adjuvant can be measured by determining the profile of cytokines secreted by T cells isolated from immunized animals, which may indicate whether Th1 or Th2 effector T cells are preferentially produced, or by assaying for functional cytotoxic T cells (see, for example, U.S. Pat. No. 6,149,922).

When used as an adjuvant in a vaccine composition, B7-H3A polypeptides or antagonists are desirably admixed as part of the vaccine composition itself. One of skill in the art of vaccine composition can readily determine suitable amounts of B7-H3A polypeptides or antagonists to adjuvant particular vaccines. Such amounts will depend upon the purpose for which the vaccine is designed, the nature of the antigen, and the dosage amounts of the antigen, as well as the species and physical and medical conditions of the vaccinate. As one example, an effective adjuvanting amount of a B7-H3A polypeptide or antagonist is desirably between about 0.01 micrograms to about 10 mg (preferably about 0.1 microgram to about 1 mg, and more preferably about 1 microgram to about 0.1 mg) of B7-H3A polypeptide or antagonist per about 25 micrograms of antigen. When administered as part of a vaccine composition, B7-H3A polypeptides or antagonists are administered by the same route as the vaccinal antigen. Any route of administration can be employed for the administration of this vaccine, e.g., subcutaneous, intraperitoneal, oral, intramuscular, intranasal and the like. The adjuvants may be given orally in alkaline solutions in vaccines appropriate for raising mucosal antibodies against antigens which give rise to intestinal diseases, as alkaline solutions such as those containing bicarbonates protect antigens and adjuvants from destruction in the upper GI tract. Alternatively, the adjuvanting effect of B7-H3A polypeptides or antagonists may be employed by administering B7-H3A polypeptides or antagonists separately from the vaccine composition, and preferably in the presence of a suitable carrier, such as saline and optionally conventional pharmaceutical agents enabling gradual release of the B7-H3A polypeptide or antagonist. The amount of the B7-H3A polypeptides or antagonists used in this mode of vaccination is similar to the ranges identified above when B7-H3A polypeptides or antagonists are part of the vaccine composition. The B7-H3A polypeptides or antagonists may be administered contemporaneously with the vaccine composition, either simultaneously therewith, or before the vaccine antigen administration. If the B7-H3A polypeptide or antagonist is administered before the vaccine composition, it is desirable to administer it about one or more days before the vaccine. When B7-H3A polypeptides or antagonists are administered as a separate component from the vaccine, they are desirably administered by the same route as the vaccinal antigen, e.g., subcutaneous route, or any other route as selected by a physician.

In addition to the administration of B7-H3A polypeptides or antagonists as an adjuvant, nucleic acid sequences encoding B7-H3A polypeptides or antagonists or a fragment thereof can also be used as an adjuvant. The nucleic acid sequences, preferably in the form of DNA, may be delivered to a vaccinate for in vivo expression of the B7-H3A polypeptide or antagonist. Naked DNA can also be used to express the B7-H3A polypeptides or antagonists in a patient (see, for example, Cohen, 1993, Science 259: 1691-1692; Fynan et al., 1993, Proc Natl Acad Sci 90: 11478-11482; and Wolff et al., 1991, Biotechniques 11: 474-485). For example, B7-H3A DNA can be incorporated into a microorganism itself, if it as a whole pathogen is to be employed as the vaccinal antigen. Alternatively, B7-H3A DNA can be administered as part of the vaccine composition or separately, but contemporaneously with the vaccine antigen, e.g., by injection. Still other modes of delivering B7-H3A polypeptide or antagonist to the vaccinate in the form of DNA are known to those of skill in the art and can be employed rather than administration of the B7-H3A polypeptide or antagonist, as desired. For example, B7-H3A DNA can be administered as part of a vector or as a cassette containing the B7-H3A DNA sequences operatively linked to a promoter sequence. When B7-H3A nucleic acid sequences are used as an adjuvant, these sequences can be operably linked to DNA sequences which encode the antigen. Hence, the vector or cassette, as described above, encoding the B7-H3A DNA sequences can additionally include sequences encoding the antigen. Each of these sequences can be operatively linked to the promoter sequence of the vector or cassette. Alternatively, naked DNA encoding the antigen can be in a separate plasmid. Where present in one or two plasmids, the naked DNA encoding the antigen and/or B7-H3A polypeptide or antagonist, upon introduction into the host cells, permits the infection of the vaccinate's cells and expression of both antigen and B7-H3A polypeptide or antagonist in vivo. When B7-H3A nucleic acid sequences are employed as the adjuvant, the amounts of DNA to be delivered and the routes of delivery may parallel the B7-H3A polypeptide or antagonist amounts and delivery described above, and can also be determined readily by one of skill in the art. Similarly the amounts of the antigen-encoding DNA can be selected by one of skill in the art.

### EXAMPLES

The following examples are intended to illustrate particular embodiments and not to limit the scope of the invention.

### Example 1

### Identification of B7-H3A, a New Member of the Human B7 Family

A cDNA library was prepared using as a template messenger RNA from CD34+ dendritic cells derived from human bone marrow, and individual cDNA clones from this library were sequenced by high-throughput sequencing. One of the clones sequenced from this library was named hh5336, and has the nucleotide sequence shown in SEQ ID NO:1. The polypeptide encoded by this clone is shown in SEQ ID NO:2. Two additional cDNA clones were isolated: the first having the complete cDNA sequence of SEQ ID NO:9 encoding a B7-H3A polypeptide having the amino acid sequence shown in SEQ ID NO:11; nucleotides 94 through 1695 of SEQ ID NO:9 encode SEQ ID NO:11, with nucleotides 1696 through 1698 corresponding to a termination codon. The B7-H3A coding sequence (nucleotides 94 through 1695 of SEQ ID NO:9) is presented as SEQ ID NO:10. These B7-H3A coding sequences were compared with publicly available preliminary human genomic DNA sequences, and the chromosome 15 contig having GenBank accession number AC022188 was identified as containing B7-H3A coding sequences. The approximate positions of the exons containing B7-H3A coding sequence in the AC022188 contig are shown in the table below, along with their locations relative to SEQ ID NOs 9 and 10. Due to the preliminary nature of assembly of sequences into the AC022188 contig, note that exons 1-7 of the B7-H3A coding sequence are in the opposite orientation to the AC022188 contig as displayed in the GenBank database entry, while exons 8 and 9 are in the same orientation as their portion of the AC022188 contig. The 5′ and 3′ untranslated regions of B7-H3A transcripts may extend further along the contig sequence beyond those portions that correspond to the indicated portions of SEQ ID NO:9, as indicated by the parentheses around the AC022188 endpoints in the table.

Corresponding positions of B7-H3A gene exons in human contig AC022188 and in cDNA sequences:

| | | Position in SEQ ID NO: 9/ |
|---|---|---|
| | Position in AC022188 | Position in SEQ ID NO: 10 |
| Exon 1 | (105255)-105123 | 40-172/1-79 |
| Exon 2 | 76829-76491 | 173-511/80-418 |
| Exon 3 | 76312-75998 | 512-826/419-733 |
| Exon 4 | 75425-75087 | 827-1165/734-1072 |
| Exon 5 | 74908-74612 | 1166-1462/1073-1369 |
| Exon 6 | 70745-70611 | 1463-1597/1370-1504 |
| Exon 7 | 69436-69395 | 1598-1639/1505-1546 |
| Exon 8 | 25236-25271 | 1640-1675/1547-1582 |
| Exon 9 | 27035-(27629) | 1676-2270/1583-1605 |

Variants of B7-H3A polypeptides are provided as naturally occurring genomic variants of the B7-H3A sequences disclosed herein; such variations can be incorporated into a B7-H3A polypeptide or nucleic acid individually or in any combination, or in combination with alternative splice variation as described below. As one example, an allelic variation involving a single change from ‘C’ to ‘A’ at position 424 of SEQ ID NO:9 or 331 of SEQ ID NO:10 produces a change from the Arg residue position 111 of SEQ ID NO:11 to a Ser residue, as for example at position 111 of SEQ ID NO:13. This variation and others are listed in the table below, and shown in Table 2:

| Amino | Position in | Nucleotide | Position in SEQ ID NO: 9/ |
|---|---|---|---|
| Acid Change | SEQ ID NO: 11 | Change | Position in SEQ ID NO: 10 |
| Pro −> Leu | 97 | C −> T | 383/290 |
| Arg −> Ser | 111 | C −> A | 424/331 |
| Val −> Phe | 134 | G −> T | 493/400 |
| Thr −> Met | 160 | C −> T | 572/479 |
| Ile −> Val | 207 | A −> G | 712/619 |
| Arg −> His | 267 | G −> A | 893/800 |
| Ala −> Thr | 279 | G −> A | 928/835 |
| Arg −> Gln | 387 | G −> A | 1253/1160 |
| Gly −> Arg | 508 | G −> A | 1615/1522 |

The amino acid sequence of B7-H3A (SEQ ID NO:11) contains two repeated sequences of 212 amino acids each. Segments of the B7-H3A (SEQ ID NO:11) amino acid sequence, each comprising one of the two repeat sequences, were compared with each other and with the amino acid sequences of these other human B7 family members—B7-H1 (GenBank AAF25807), PD-L2 (GenBank AF344424), and B7h (also called GL50, GenBank AAF34739) (SEQ ID NO:14-SEQ ID NO:16, respectively)—using the GCG “pretty” multiple sequence alignment program, with amino acid similarity scoring matrix=blosum62, gap creation penalty=8, and gap exten-sion penalty=2. An alignment of these sequences is shown in Table 1, and includes consensus residues which are identical among at least three of the amino acid sequences in the alignment. The numbering of residues in the alignment is shown by reference to the SEQ ID NO:11 repeat 1 sequence (top set of numbers) and to the SEQ ID NO:11 repeat 2 sequence (bottom set of numbers). The capitalized residues in the alignment are those which match the consensus residues. Amino acid sub-stitutions and other alterations (deletions, insertions, etc.) to B7-H3A amino acid sequences (e.g. SEQ ID Nos 11 and 13) are predicted to be more likely to alter or disrupt B7-H3A polypeptide activities if they result in changes to the capitalized residues of the amino acid sequences as shown in Table 1, and particularly if those changes do not substitute an amino acid of similar structure (such as substitution of any one of the aliphatic residues—Ala, Gly, Leu, Ile, or Val—for another aliphatic residue), or a residue present in other B7 polypeptides at that conserved position. Conversely, if a change is made to a B7-H3A amino acid sequence resulting in substitution of the residue at that position in the alignment from one of the other Table 1 B7 polypeptide sequences, it is less likely that such an alteration will affect the function of the altered B7-H3A polypeptide. For example, the consensus residue at position 48/260 in Table 1 is valine, and one of the B7 polypeptides (B7-H1) has a tyrosine residue at that position. Substitution of tyrosine or one of the residues that are chemically similar to valine—one of the other aliphatic residues—for valine at that position is less likely to alter the function of the polypeptide than substitution of aspartate or glutamine, etc. Embodiments of the invention include B7-H3A polypeptides and fragments of B7-H3A polypeptides, comprising altered amino acid sequences. Altered B7-H3A polypeptide sequences share at least 30%, or more preferably at least 40%, or more preferably at least 50%, or more preferably at least 55%, or more preferably at least 60%, or more preferably at least 65%, or more preferably at least 70%, or more preferably at least 75%, or more preferably at least 80%, or more preferably at least 85%, or more preferably at least 90%, or more preferably at least 95%, or more preferably at least 97.5%, or more preferably at least 99%, or most preferably at least 99.5% amino acid identity with one or more of the B7 amino acid sequences shown in Table 1.

TABLE 1

Alignment of B7-H3A amino acid sequence with those of
other B7 polypeptides

[symbol]

: B7-H3A signal peptide
C: conserved cysteine [symbol]
: Ig V-like domain [symbol]
: Ig C-like domain
Bold Italics: B7-H3A transmembrane domain

SEQ
ID

NO:

| | | |
|---|---|---|
| | | 1 50 |
| | | 239 268 |
| Hs B7H-1 | 14 | ˜˜˜˜˜˜˜˜˜˜ mrifavfifm tywhLlnAft VtVPkDlyVv eyGSnmTieC |
| Hs PD-L2 | 15 | ˜˜˜˜˜˜˜˜mi flllmlslel qlhqiaalft VtVPkelyii ehGSnvTLeC |
| B73A rpt1 | 11¹ | [symbol] mlrrrgspgm gvhvgaalga lwfcLtgAle VqVPEDpVVA lVGtDaTLcC |
| B73A rpt2 | 11² | ˜˜˜˜˜˜˜˜˜˜ ˜˜˜˜˜˜˜˜˜˜ pqrsptgAve VqVPEDpVVA lVGtDaTLrC |
| Hs B7h | 16 | ˜˜˜˜˜˜˜˜˜˜ ˜˜˜mrlgspg llflLfsslr adtqEkeVrA mVGSDveLsC |
| consensus | | ---------- ---------- ----L--A- [symbol] - V-VPED-VVA -VGSD-TL-C |
| | | 51 94 |
| | | 269 312 |
| Hs B7H-1 | 14 | kFpvEkqldL AaLivyWeme Dkniiqfvhg eedlkv.... QhSsYRqRAr |
| Hs PD-L2 | 15 | nFdtgshvnL gaita..... ...sLqkv.. endt...... ..SphReRAt |
| B73A rpt1 | 11¹ | sFspEpgFsL AqLnliWQlt DtKqLV.... ..hSfaEgqD QgSaYaNRta |
| B73A rpt2 | 11² | sFspEpgFsL AqLnliWQlt DtKqLV.... ..hSftEgrD QgSaYaNRta |
| Hs B7h | 16 | acpegsrFdL ndvyvyWQts esKtvVtyhi pqnSslEnvD ..SrYRNRAl |
| consensus | | [symbol] -F--E--F-L A-L---WQ-- D-K-LV---- ---S--E--D Q-S-YRNRA- |
| | | 95 139 |
| | | 313 357 |
| Hs B7H-1 | 14 | LlkDqLslGN AaLqitdVkl qDaGvyrCmi S...yGga.d yk.riTvkVn |
| Hs PD-L2 | 15 | LleeqLplGk ASfhipqVqV rDEGqyqCii i...yGvawd yk.ylTLkVk |
| B73A rpt1 | 11¹ | LfPDlLaqGN ASLRLqrVrV aDEGsFtCfV SirdfG.... .SaaVsLqVA |
| B73A rpt2 | 11² | LfPDlLaqGN ASLRLqrVrV aDEGsFtCfV SirdfG.... .SaaVsLqVA |
| Hs B7h | 16 | msPagmlrGd fSLRLfnVtp qDEqkFhClV lsqslGfqev lSveVTLhVA |
| consensus | | [symbol] L-PD-L--GN ASLRL--V-V -DEG-F-C-V S----G---- -S--VTL-VA |
| | | 140 189 |
| | | 358 407 |
| Hs B7H-1 | 14 | APYnKinqri lvvdPvt..s EhelTCqa.e GYPkAEViWt ssdhqvLsGk |
| Hs PD-L2 | 15 | AsYrKinthi lkv.Pet..D EVelTCqa.t GYPlAEVsWp n.....vsvp |
| B73A rpt1 | 11¹ | APYSKPsmtl ePnkdlrpgD tVTiTCsSyq GYPeAEVfWq dgqgvpLtGn |
| B73A rpt2 | 11² | APYSKPsmtl ePnkdlrpgD tVTiTCsSyr GYPeAEVfWq dgQgvpLtGn |
| Hs B7h | 16 | AnfSvPvvs. aPhsPsq..D ElTfTCtSin GYPrpnVyWi nktdnsLldq |
| consensus | | [symbol] APYSKP---- -P--P----D EVT-TC-S-- GYP-AEV-W- ------L-G- |
| | | 190 236 |
| | | 408 454 |
| Hs B7H-1 | 14 | tTTtnsk..r EekLFnVtSt LRintttNei fyCtfRrldp eenht.aelv |
| Hs PD-L2 | 15 | anTShsr..t peGLyqVtSV LRlkpppgrn fSCvf..... wnthv.relT |
| B73A rpt1 | 11¹ | vTTSq..maN EqGLFDVhSi LRvvlgaNgt ySClvRNpvL QQd.ahsSvT |
| B73A rpt2 | 11² | vTTSq..maN EqGLFDVhSV LRvvlgaNgt ySClvRNpvL QQd.ahgSvT |
| Hs B7h | 16 | alqndtvflN mrGLyDVvSV LRiartpsvn igCcieNvlL QQnltvgSqT |
| consensus | | [symbol] -TTS-----N E-GLFDV-SV LR-----N-- -SC--RN--L QQ-----S-T |
| | | 237 244 |
| | | 455 493 |
| Hs B7H-1 | 14 | ipelplahpp nerthlvilg aill..clgv altfI...fr LrkgrmmdvK |
| Hs PD-L2 | 15 | lasidlqsqm eprthptwll hifipsci.i afifIatviA Lrk..qlcqK |
| B73A rpt1 | 11¹ | .........I T..Pqrs..p t˜˜˜˜˜˜˜˜˜ ˜˜˜˜˜˜˜˜˜˜ ˜˜˜˜˜˜˜˜˜˜ |
| B73A rpt2 | 11² | .........I TgqPmtf..p pealwvtvgl svclIallva LafvcwrkiK |
| Hs B7h | 16 | gndigerdkI TenPvstgek naatwsilav lcllvvvavA igwvcrdrcl |
| consensus | | [symbol] ---------I T--P------ ---------- ----I----A L--------K |
| | | 494 534 |
| Hs B7H-1 | 14 | kcgiqdtnsk kqsdThlEet ˜˜˜˜˜˜˜˜˜˜ ˜˜˜˜˜˜˜˜˜˜ ˜ |
| Hs PD-L2 | 15 | lysskdttkr pvttTkrEvn sai˜˜˜˜˜˜˜ ˜˜˜˜˜˜˜˜˜˜ ˜ |
| B73A rpt2 | 11² | qsceeenaga edqdgegEgs ktalqplkhs dskeddgqei a |
| Hs B7h | 16 | qh.syagawa vspeTeltes wnllllls˜˜ ˜˜˜˜˜˜˜˜˜˜ ˜ |
| consensus | | ---------- ----T--E-- ---------- ---------- - |

¹′B73A rpt1′ is amino acids 1-244 of SEQ ID NO:11
²′B73A rpt2′ is amino acids 239-534 of SEQ ID NO:11

The amino acid sequence of the B7-H3A polypeptides of SEQ ID NO:11 and SEQ ID NO:13 were also compared to each other and to the amino acid sequences of the following similar human polypeptides identified through searches of public databases: “amyloid precursor protein protease” (WO 00/68266), “secretory or membrane protein” PSEC0249 (EP 1067182 A2), B7 homolog 3 (B7-H3) (GenBank NP_{—}079516), and PRO352 (GeneSeq Y41705) (SEQ ID Nos 17-19 and SEQ ID NO:4, respectively); the results of this comparison are shown in Table 2. The B7-H3 and PRO352 polypeptides lack the N-terminal C-like Ig domain and the C-terminal V-like Ig domain relative to the B7-H3A polypeptides of SEQ ID NO:11 and SEQ ID NO:13, and are apparently translated from an alternatively spliced form of “B7-H3” mRNA that does not contain exons 3 and 4 of the B7-H3A coding sequence. The “amyloid precursor protein protease” (disclosed in the patent publication WO 00/68266) was only described in terms of having protease activity, and while the “secretory or membrane protein” PSEC0249 (disclosed in the patent publication EP 1067182 A2) was noted to have some sequence similarity to “butryophilin precursor”, no description was provided of the specific biological function or utility of the PSEC0249 polypeptide. An additional patent publication, WO 01/18204-A1, has been identified which discloses polypeptides having amino acid sequences similar to B7-H3A; however, the B7-H3A polypeptide sequences of the present invention were apparently the first to be identified as having the N-terminal V-like and C-like Ig domains of SEQ ID Nos 2, 11, and 13, and possessing T cell costimulatory activity.

TABLE 2

Alignment of B7-H3A amino acid sequence with those of
B7-H3-like polypeptides

[symbol]

: signal peptide
C: conserved cysteine [symbol]
: Ig V-like domain
bold lowercase: amino acid variation [symbol]
: Ig C-like domain
Bold Italics: transmembrane domain

SEQ
ID

NO:

| | | |
|---|---|---|
| | | 1 50 |
| Hs B7-H3A | 11 | MLRRRGSPGMGVHVGAALGALWFCLTGALEVQVPEDPVVALVGTDATLCC |
| Hs WO 0068266 | 17 | MLRRRGSPGMGVHVGAALGALWFCLTGALEVQVPEDPVVALVGTDATLCC |
| Hs PSEC0249 | 18 | MLRRRGSPGMGVHVGAALGALWFCLTGALEVQVPEDPVVALVGTDATLCC |
| Hs B7-H3A 8b | 13 | MLRRRGSPGMGVHVGAALGALWFCLTCALEVQVPEDPVVALVGTDATLCC |
| Hs B7-H3 | 19 | MLRRRGSPGMGVHVGAALGALWFCLTGALEVQVPEDPVVALVGTDATLCC |
| Hs PRO352 | 4 | [symbol] MLRRRGSPGMGVHVGAALGALWFCLTGA [symbol] LEVQVPEDPVVALVGTDATLCC |

←-- repeat 1 --. . .

| | | |
|---|---|---|
| | | 51 100 |
| Hs B7-H3A | 11 | SFSPEPGFSLAQLNLIWQLTDTKQLVHSFAEGQDQGSAYANRTALFPDLL |
| Hs WO 0068266 | 17 | SFSPEPGFSLAQLNLIWQLTDTKQLVHSFAEGQDQGSAYANRTALFPDLL |
| Hs PSEC0249 | 18 | SFSPEPGFSLAQLNLIWQLTDTKQLVHSFAEGQDQGSAYANRTALFPDLL |
| Hs B7-H3A 8b | 13 | SFSPEPGFSLAQLNLIWQLTDTKQLVHSFAEGQDQGSAYANRTALF1DLL |
| Hs B7-H3 | 19 | SFSPEPGFSLAQLNLIWQLTDTKQLVHSFAEGQDQGSAYANRTALFPDLL |
| Hs PRO352 | 4 | [symbol] SFSPEPGFSLAQLNLIWQLTDTKQLVHSFAEGQDQGSAYANRTALFPDLL |
| | | . . . -- repeat 1 -- . . . |
| | | 101 150 |
| Hs B7-H3A | 11 | AQGNASLRLQRVRVADEGSFTCFVSIRDFGSAAVSLQVAAPYSKPSMTLE |
| Hs WO 0068266 | 17 | AQGNASLRLQRVRVADEGSFTCFVSIRDFGSAAVSLQVAAPYSKPSMTLE |
| Hs PSEC0249 | 18 | AQGNASLRLQRVRVADEGSFTCFVSIRDFGSAAVSLQVAAPYSKPSMTLE |
| Hs B7-H3A 8b | 13 | AQGNASLRLQsVRVADEGSFTCFVSIRDFGSAAVSLQVAAPYSKPSMTLE |
| Hs B7-H3 | 19 | AQGNASLRLQRVRVADEGSFTCFVSIRDFGSAAVSLQVA........... |
| Hs PRO352 | 4 | [symbol] AQGNASLRLQRVRVADEGSFTCFVSIRDFGSAAVSLQVA........... |
| | | . . . -- repeat 1 -- . . . 139 |
| | | 151 200 |
| Hs B7-H3A | 11 | PNKDLRPGDtVTITCSSYQGYPEAEVFWQDGQGVPLTGNVTTSQMANEQG |
| Hs WO 0068266 | 17 | PNKDLRPGDtVTITCSSYQGYPEAEVFWQDGQGVPLTGNVTTSQMANEQG |
| Hs PSEC0249 | 18 | PNKDLRPGDmVTITCSSYQGYPEAEVFWQDGQGVPLTGNVTTSQMANEQG |
| Hs B7-H3A 8b | 13 | [symbol] PNKDLRPGDmVTITCSSYQGYPEAEVFWQDGQGVPLTGNVTTSQMANEQG |
| | | . . . -- repeat 1 -- . . . |
| | | 201 250 |
| Hs B7-H3A | 11 | LFDVHSILRVVLGANGTYSCLVRNPVLQQDAHSSVTITPQRSPTGAVEVQ |
| Hs WO 0068266 | 17 | LFDVHSILRVVLGANGTYSCLVRNPVLQQDAHSSVTITPQRSPTGAVEVQ |
| Hs PSEC0249 | 18 | LFDVHSILRVVLGANGTYSCLVRNPVLQQDAHSSVTITPQRSPTGAVEVQ |
| Hs B7-H3A 8b | 13 | [symbol] LFDVHSILRVVLGANGTYSCLVRNPVLQQDAHSSVTITPQRSPT [symbol] GAVEVQ |
| | | . . . -- repeat 1 --→ ← rpt2 |
| | | 251 300 |
| Hs B7-H3A | 11 | VPEDPVVALVGTDATLRCSFSPEPGFSLAQLNLIWQLTDTKQLVHSFTEG |
| Hs WO 0068266 | 17 | VPEDPVVALVGTDATLRCSFSPEPGFSLAQLNLIWQLTDTKQLVHSFTEG |
| Hs PSEC0249 | 18 | VPEDPVVALVGTDATLRCSFSPEPGFSLAQLNLIWQLTDTKQLVHSFTEG |
| Hs B7-H3A 8b | 13 | [symbol] VPEDPVVALVGTDATLhCSFSPEPGFSLtQLNLIWQLTDTKQLVHSFTEG |
| | | . . . -- repeat 2 -- . . . |
| | | 301 350 |
| Hs B7-H3A | 11 | RDQGSAYANRTALFPDLLAQGNASLRLQRVRVADEGSFTCFVSIRDFGSA |
| Hs WO 0068266 | 17 | RDQGSAYANRTALFPDLLAQGNASLRLQRVRVADEGSFTCFVSIRDFGSA |
| Hs PSEC0249 | 18 | RDQGSAYANRTALFPDLLAQGNASLRLQRVRVADEGSFTCFVSIRDFGSA |
| Hs B7-H3A 8b | 13 | [symbol] RDQGSAYANRTALFPDLLAQGNASLRLQRVRVADEGSFTCFVSIRDFGSA |
| | | . . . -- repeat 2 -- . . . |
| | | 351 400 |
| Hs B7-H3A | 11 | AVSLQVAAPYSKPSMTLEPNKDLRPGDTVTITCSSYRGYPEAEVFWQDGQ |
| Hs WO 0068266 | 17 | AVSLQVAAPYSKPSMTLEPNKDLRPGDTVTITCSSYRGYPEAEVFWQDGQ |
| Hs PSEC0249 | 18 | AVSLQVAAPYSKPSMTLEPNKDLRPGDTVTITCSSYRGYPEAEVFWQDGQ |
| Hs B7-H3A 8b | 13 | AVSLQVAAPYSKPSMTLEPNKDLRPGDTVTITCSSYRGYPEAEVFWQDGQ |
| Hs B7-H3 | 19 | APYSKPSMTLEPNKDLRPGDTVTITCSSYRGYPEAEVFWQDGQ |
| Hs PRO352 | 4 | [symbol] .......APYSKPSMTLEPNKDLRPGDTVTITCSSYqGYPEAEVFWQDGQ |
| | | 140 182 |
| | | . . . -- repeat 2 -- . . . |
| | | 401 450 |
| Hs B7-H3A | 11 | GVPLTGNVTTSQMANEQGLFDVHSVLRVVLGANGTYSCLVRNPVLQQDAH |
| Hs WO 0068266 | 17 | GVPLTGNVTTSQMANEQGLFDVHSVLRVVLGANGTYSCLVRNPVLQQDAH |
| Hs PSEC0249 | 18 | GVPLTGNVTTSQMANEQGLFDVHSVLRVVLGANGTYSCLVRNPVLQQDAH |
| Hs B7-H3A 8b | 13 | GVPLTGNVTTSQMANEQGLFDVHSVLRVVLGANGTYSCLVRNPVLQQDAH |
| Hs B7H3 | 19 | GVPLTGNVTTSQMANEQGLFDVHSVLRVVLGANGTYSCLVRNPVLQQDAH |
| Hs PRO352 | 4 | G [symbol] VPLTGNVTTSQMANEQGLFDVHSVLRVVLGANGTYSCLVRNPVLQQDAH |
| | | 183 232 |
| | | . . . -- repeat 2 -- . . . |
| | | 451 500 |
| Hs B7-H3A | 11 | GSVTITGQPMTFPPEALWVTVGLSVCLIALLVALAFVCWRKIKQSCEEEN |
| Hs WO 0068266 | 17 | GSVTITGQPMTFPPEALWVTVGLSVCLIALLVALAFVCWRKIKQSCEEEN |
| Hs PSEC0249 | 18 | GSVTITGQPMTFPPEALWVTVGLSVCLIALLVALAFVCWRKIKQSCEEEN |
| Hs B7-H3A 8b | 13 | GSVTITGQPMTFPPEALWVTVGLSVCLIALLVALAFVCWRKIKQSCEEEN |
| Hs B7-H3 | 19 | GSVTITGQPMTFPPEALWVTVGLSVCLIALLVALAFVCWRKIKQSCEEEN |
| Hs PRO352 | 4 | [symbol] xSVTITGQPMTFPPEALWVTVGLSVCLIALLVALAFVCWRKIKQSCEEEN |
| | | 233 282 |
| | | . . . rpt2→ |
| | | 501 534 |
| Hs B7-H3A | 11 | AGAEDQDGECEGSKTALQPLKHSDSKEDDGQEIA |
| Hs WO 0068266 | 17 | AGAEDQDGEGEGSKTALQPLKHSDSKEDDGQEIA |
| Hs PSEC0249 | 18 | AGAEDQDGEGEGSKTALQPLKHSDSKEDDGQEIA |
| Hs B7-H3A 8b | 13 | AGAEDQDrEGEGSKTALQPLKHSDSKEDDGQEIA |
| Hs B7-H3 | 19 | AGAEDQDGEGEGSKTALQPLKHSDSKEDDGQEIA |
| Hs PRO352 | 41 | AGAEDQDGEGEGSKTALQPLKHSDSKEDDGQEIA |
| | | 283 316 |

### Example 2

### Chromosome Mapping

The genomic location of the B7-H3A “hh5336” cDNA clone was identified using PCR-based mapping strategies. Initial human chromosomal assignments were made using hh5336-specific PCR primers and a BIOS Somatic Cell Hybrid PCRable DNA kit from BIOS Laboratories (New Haven, Conn.), following the manufacturer's instructions. The hh5336 sequences mapped to human chromosome 15. More detailed mapping was performed using a Genebridge 4 Radiation Hybrid Panel (Research Genetics, Huntsville, Ala.; described in Walter, Mass. et al., Nature Genetics 7:22-28, 1994). Data from this analysis was then submitted electronically to the MIT Radiation Hybrid Mapper (www-genome.wi.mit.edu/cgi-bin/contig/rhmapper.pl) following the instructions contained therein. This analysis yielded specific genetic marker names which, when submitted electronically to the NCBI Genemap browser (www.ncbi.nlm.nih.gov/genemap/map.cgi?CHR=15), yielded the specific chromosome 19 interval. This analysis showed that hh5336 mapped 5.55 cR distal to marker WI-6247, which is located at 249.12 cR from the top of chromosome 15 on the Genebridge 4 map. The B7-H3A “hh5336” cDNA clone thus mapped to human chromosome 15q22, at the position 254.67 cR, or 71.4 cM, from the top of chromosome 15.

### Example 3

### Monoclonal Antibodies That Bind Polypeptides of the Invention

This example illustrates a method for preparing monoclonal antibodies that bind B7-H3A polypeptides. Other conventional techniques can be used, such as those described in U.S. Pat. No. 4,411,993. Suitable immunogens that may be employed in generating such antibodies include, but are not limited to, purified B7-H3A polypeptide, an immunogenic fragment thereof, and cells expressing high levels of B7-H3A polypeptide or an immunogenic fragment thereof. DNA encoding a B7-H3A polypeptide can also be used as an immunogen, for example, as reviewed by Pardoll and Beckerleg in Immunity 3: 165, 1995.

Rodents (BALB/c mice or Lewis rats, for example) are immunized with B7-H3A polypeptide immunogen emulsified in an adjuvant (such as complete or incomplete Freund's adjuvant, alum, or another adjuvant, such as Ribi adjuvant R700 (Ribi, Hamilton, Mont.)), and injected in amounts ranging from 10-100 micrograms subcutaneously or intraperitoneally. DNA can be given intradermally (Raz et al., 1994, Proc. Natl. Acad. Sci. USA 91: 9519) or intamuscularly (Wang et al., 1993, Proc. Natl. Acad. Sci. USA 90: 4156); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision to test for B7-H3A polypeptide antibodies by dot-blot assay, ELISA (enzyme-linked immunosorbent assay), immunoprecipitation, or other suitable assays, such as FACS analysis of inhibition of binding of B7-H3A polypeptide to a B7-H3A polypeptide binding partner. Following detection of an appropriate antibody titer, positive animals are provided one last intravenous injection of B7-H3A polypeptide in saline. Three to four days later, the animals are sacrificed, and spleen cells are harvested and fused to a murine myeloma cell line, e.g., NS1 or preferably P3X63Ag8.653 (ATCC CRL-1580). These cell fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells can be screened by ELISA for reactivity against purified B7-H3A polypeptide by adaptations of the techniques disclosed in Engvall et al., (Immunochem. 8: 871, 1971) and in U.S. Pat. No. 4,703,004. A preferred screening technique is the antibody capture technique described in Beckmann et al., (J. Immunol. 144: 4212, 1990). Positive hybridoma cells can be injected intraperitoneally into syngeneic rodents to produce ascites containing high concentrations (for example, greater than 1 milligram per milliliter) of anti-B7-H3A polypeptide monoclonal antibodies. Alternatively, hybridoma cells can be grown in vitro in flasks or roller bottles by various techniques. Monoclonal antibodies can be purified by ammonium sulfate precipitation, followed by gel exclusion chromatography. Alternatively, affinity chromatography based upon binding of antibody to protein A or protein G can also be used, as can affinity chromatography based upon binding to B7-H3A polypeptide.

### Example 4

### Antisense Inhibition of B7-H3A Nucleic Acid Expression

In accordance with the present invention, a series of oligonucleotides are designed to target different regions of the B7-H3A mRNA molecule, using the nucleotide sequence of SEQ ID NO:9 as the basis for the design of the oligonucleotides. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule.

The oligonucleotides can be oligodeoxynucleotides, with phosphorothioate backbones (internucleoside linkages) throughout, or can have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites can be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyl-uridine, 5′-O-tert-butyldiphenylsilyl-2′-O-[(2-formadoximinooxy)ethyl]-5-methyluridine, 5′-O-tert-butyldiphenylsilyl-2′-O-[N,N-dimethylaminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-[(2-cyanoethyl)-N,N-diiso-propylphosphoramidite].

Modified oligonucleosides can also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P=O or P=S linkages, which are prepared as described in U.S. Pat. Nos. 5,378,825, 5,386,023, 5,489,677, 5,602,240 and 5,610,289.

Formacetal- and thioformacetal-linked oligonucleosides can also be used and are prepared as described in U.S. Pat. Nos. 5,264,562 and 5,264,564; and ethylene oxide linked oligonucleosides can also be used and are prepared as described in U.S. Pat. No. 5,223,618. Peptide nucleic acids (PNAs) can be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]—[2′-deoxy]—[2′-O-Me] chimeric phosphorothioate oligonucleotides, [2′-O-(2-methoxyethyl)]—[2′-deoxy]—[2′-O-(methoxyethyl)] chimeric phosphorothioate oligonucleotides, and [2′-O-(2-methoxy-ethyl)phosphodiester]—[2′-deoxy phosphoro-thioate]—[2′-O-(2-methoxyethyl)phosphodiester] chimeric oligonucleotides, all of which can be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleotides/oligonucleosides are synthesized according to U.S. Pat. No. 5,623,065.

Oligonucleotides are preferably synthesized via solid phase P(III) phosphoramidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of B7-H3A nucleic acid expression can be assayed in a variety of ways known in the art. For example, B7-H3A mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. B7-H3A protein levels can be quantitated in a variety of ways well known in the art, such as immunoprecipitation, Western blot analysis (immunoblotting), ELISA, or fluorescence-activated cell sorting (FACS). Antibodies directed to B7-H3A polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

Sequences Presented in the Sequence Listing

| SEQ ID NO | Type | Description |
|---|---|---|
| SEQ ID NO: 1 | Nucleotide | Portion of human B7-H3A nucleic |
| | | acid sequence (‘hh5336’) |
| SEQ ID NO: 2 | Amino acid | Portion of human B7-H3A amino |
| | | acid sequence (‘hh5336’) |
| SEQ ID NO: 3 | Nucleotide | Human PRO352 nucleic acid |
| | | sequence (GeneSeq Z34006) |
| SEQ ID NO: 4 | Amino acid | Human PRO352 amino acid |
| | | sequence (GeneSeq Y41705) |
| SEQ ID NO: 5 | Nucleotide | Oligonucleotide primer for human |
| | | B7-H3A nucleic acid sequences |
| SEQ ID NO: 6 | Nucleotide | Oligonucleotide primer for human |
| | | B7-H3A nucleic acid sequences |
| SEQ ID NO: 7 | Nucleotide | Oligonucleotide primer for human |
| | | B7-H3A nucleic acid sequences |
| SEQ ID NO: 8 | Amino acid | FLAG peptide amino acid sequence |
| SEQ ID NO: 9 | Nucleotide | Human B7-H3A nucleic acid |
| | | sequence (cDNA sequence) |
| SEQ ID NO: 10 | Nucleotide | Human B7-H3A nucleic acid |
| | | sequence (coding portion of cDNA) |
| SEQ ID NO: 11 | Amino acid | Human B7-H3A amino acid sequence |
| SEQ ID NO: 12 | Nucleotide | Variant human B7-H3A “8b” |
| | | nucleic acid sequence (coding |
| | | sequence) |
| SEQ ID NO: 13 | Amino acid | Variant human B7-H3A “8b” |
| | | amino acid sequence |
| SEQ ID NO: 14 | Amino acid | Human B7-H1 amino acid sequence |
| | | (GenBank AAF25807) |
| SEQ ID NO: 15 | Amino acid | Human PD-L2 amino acid sequence |
| | | (GenBank AF344424) |
| SEQ ID NO: 16 | Amino acid | Human B7h (GL50) amino acid |
| | | sequence (GenBank AAF34739) |
| SEQ ID NO: 17 | Amino acid | Human “amyloid precursor |
| | | protein protease” |
| | | (WO 00/68266) |
| SEQ ID NO: 18 | Amino acid | Human “secretory or membrane |
| | | protein” PSEC0249 |
| | | (EP 1067182 A2) |
| SEQ ID NO: 19 | Amino acid | Human B7 homolog 3 (B7-H3) |
| | | (GenBank NP_079516) |
| SEQ ID NO: 20 | Amino acid | Fusion protein: amino acids |
| | | 1-238 of SEQ ID NO: 13 |
| | | fused to IgG Fc |

What is claimed is:

1. An isolated polypeptide comprising an amino acid sequence selected from the group consisting of:
(a) the amino acid sequence of SEQ ID NO:11;
(b) the amino acid sequence of SEQ ID NO:13;
(c) the amino acid sequence of SEQ ID NO:2;
(d) an amino acid sequence selected from the group consisting of:
(d1) amino acids 27 through 169 of SEQ ID NO:2;
(d2) amino acids 29 through 387 of SEQ ID NO:2;
(d3) amino acids 29 through 387 of SEQ ID NO:2 and further comprising amino acids 250 through 270 of SEQ ID NO:4;
(d4) amino acids 29 through 387 of SEQ ID NO:2 and further comprising amino acids 273 through 316 of SEQ ID NO:4;
(d5) amino acids Xaa1 through 139 of SEQ ID NO:11, wherein Xaa1 is an amino acid selected from the group consisting of amino acids 27 through 29 of SEQ ID NO:11; and
(d6) amino acids 140 through Xaa2 of SEQ ID NO:11, wherein Xaa2 is an amino acid selected from the group consisting of amino acids 238 through 244 of SEQ ID NO:11;
(e) a fragment of the amino acid sequences of any of (a)-(d) comprising at least 20 contiguous amino acids, wherein a polypeptide consisting of said fragment has T cell immunomodulatory activity;
(f) a fragment of the amino acid sequences of any of (a)-(d), wherein a polypeptide consisting of said fragment has T cell immunomodulatory activity;
(g) a fragment of the amino acid sequences of any of (a)-(d) comprising B7-H3A extracellular domain amino acid sequences;
(h) an amino acid sequence comprising at least 20 amino acids and sharing amino acid identity with the amino acid sequences of any of (a)-(g), wherein the percent amino acid identity is selected from the group consisting of: at least 70%, at least 75%, at least 80%, at least 85%, at least 90%, at least 95%, at least 97.5%, at least 99%, and at least 99.5%, and wherein a polypeptide consisting of said amino acid sequence has T cell immunomodulatory activity;
(i) an amino acid sequence of (h), wherein a polypeptide comprising said amino acid sequence of (h) binds to an antibody that also binds to a polypeptide comprising an amino acid sequence of any of (a)-(g); and wherein wherein a polypeptide consisting of said amino acid sequence has T cell immunomodulatory activity; and
(j) allelic variants of (a)-(i) above.

2. An isolated nucleic acid encoding a polypeptide of claim 1.

3. The nucleic acid of claim 2 comprising a nucleotide sequence selected from the group consisting of:
(a) SEQ ID NO:1;
(b) SEQ ID NO:10;
(c) SEQ ID NO:12;
(d) a nucleotide sequence encoding an amino acid sequence selected from the group consisting of:
(d1) amino acids 27 through 169 of SEQ ID NO:2;
(d2) amino acids 29 through 387 of SEQ ID NO:2;
(d3) amino acids 29 through 387 of SEQ ID NO:2 and further comprising amino acids 250 through 270 of SEQ ID NO:4;
(d4) amino acids 29 through 387 of SEQ ID NO:2 and further comprising amino acids 273 through 316 of SEQ ID NO:4;
(d5) amino acids Xaa1 through 139 of SEQ ID NO:11, wherein Xaa1 is an amino acid selected from the group consisting of amino acids 27 through 29 of SEQ ID NO:11; and
(d6) amino acids 140 through Xaa2 of SEQ ID NO:11, wherein Xaa2 is an amino acid selected from the group consisting of amino acids 238 through 244 of SEQ ID NO:11;
(e) allelic variants of (a)-(d).

4. An isolated genomic nucleic acid corresponding to the nucleic acid of claim 2.

5. An isolated nucleic acid, having a length of at least 15 nucleotides, that hybridizes under conditions of moderate stringency to the nucleic acid of claim 2.

6. An isolated nucleic acid comprising a nucleotide sequence that shares nucleotide sequence identity with the nucleotide sequences of the nucleic acids of claim 2, wherein the percent nucleotide sequence identity is selected from the group consisting of: at least 70%, at least 75%, at least 80%, at least 85%, at least 90%, at least 95%, at least 97.5%, at least 99%, and at least 99.5%, wherein the nucleic acid encodes a polypeptide having T cell stimulatory activity.

7. An expression vector comprising at least one nucleic acid according to claim 2.

8. A recombinant host cell comprising at least one nucleic acid according to claim 2.

9. The recombinant host cell of claim 8, wherein the nucleic acid is integrated into the host cell genome.

10. A process for producing a polypeptide encoded by the nucleic acid of claim 2, comprising culturing a recombinant host cell under conditions promoting expression of said polypeptide, wherein the recombinant host cell comprises at least one nucleic acid according to claim 2.

11. The process of claim 10 further comprising purifying said polypeptide.

12. The polypeptide produced by the process of claim 11.

13. An isolated antibody that binds to the polypeptide of claim 12.

14. The antibody of claim 13 wherein the antibody is a monoclonal antibody.

15. The antibody of claim 13 wherein the antibody inhibits the activity of the polypeptide of claim 12.

16. A method for identifying compounds that alter B7-H3A polypeptide activity comprising
(a) mixing a test compound with the polypeptide of any of claim 12; and
(b) determining whether the test compound alters the B7-H3A polypeptide activity of said polypeptide.

17. A method for identifying compounds that inhibit the binding activity of B7-H3A polypeptides comprising
(a) mixing a test compound with the polypeptide of claim 12 and a binding partner of said polypeptide; and
(b) determining whether the test compound inhibits the binding activity of said polypeptide.

18. A method for increasing T cell immunomodulatory activity comprising providing at least one compound selected from the group consisting of the polypeptide of claim 12 and agonists of said polypeptides.

19. A method for decreasing T cell immunomodulatory activity comprising providing at least one antagonist of the polypeptide of claim 12.

20. The method of claim 19 wherein the antagonist is an antibody that inhibits the activity of said polypeptide.


SEQUENCE LISTING (20 sequences)

SEQ ID NO: 1 - DNA, 1206 bp, Homo sapiens
ccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccttcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggg

SEQ ID NO: 2 - protein, 387 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAFSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYR

SEQ ID NO: 3 - DNA, 1998 bp, Homo sapiens
cgggccgcccccggcccccattcgggccgggcctcgctgcggcggcgactgagccaggctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacrgctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttcccctccttcctccctgccccaagtgaagacagggcagggccaggaatgctttggggacaccgaggggactgccccccacccccaccatggtgctattctggggctggggcagtcttttcctggcttgcctctggccagctcctggcctctggtagagtgagacttcagacgttctgatgccttccggatgtcatctctccctgccccaggaatggaagatgtgaggacttctaatttaaatgtgggactcggagggattttgtaaactgggggtatattttggggaaaataaatgtctttgtaaaaaaaaaaaaaaaaaaaaaa

SEQ ID NO: 4 - protein, 316 aa, Homo sapiens
  Unsure (233)..(233): unsure
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSVLRVVLGANGTYSCLVRNPVLQQDAHXSVTITGQPMTFPPEALWVTVGLSVCLIALLVALAFVCWRKIKQSCEEENAGAEDQDGEGEGSKTALQPLKHSDSKEDDGQEIA

SEQ ID NO: 5 - DNA, 26 bp, Artificial Sequence
  oligonucleotide primer
cacagtttcaccgaaggccgggacca

SEQ ID NO: 6 - DNA, 24 bp, Artificial Sequence
  oligonucleotide primer
tgaggacccggtggtggccctagt

SEQ ID NO: 7 - DNA, 18 bp, Artificial Sequence
  oligonucleotide primer
ccccagagaagccccaca

SEQ ID NO: 8 - protein, 8 aa, Artificial Sequence
  FLAG peptide
DYKDDDDK

SEQ ID NO: 9 - DNA, 2270 bp, Homo sapiens
ctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttc

SEQ ID NO: 10 - DNA, 1605 bp, Homo sapiens
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctga

SEQ ID NO: 11 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 12 - DNA, 1605 bp, Homo sapiens
atgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcctggacctgctggcacagggcaacgcatccctgaggctgcagagcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggcccggggacatggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcactgctccttctcccccgagcctggcttcagcctgacacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacatgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctctctgtctgtctcattgcactgctggtggccctggctttcgtgtgctggagaaagatcaaacagagctgtgaggaggagaatgcaggagccgaggaccaggatagggagggagaaggctccaagacagccctgcagcctctgaaacactctgacagcaaagaagatgatggacaagaaatagcctga

SEQ ID NO: 13 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFLDLLAQGNASLRLQSVRVADEGSFTCFVSIRDFGSAAVSLQVAAPYSKPSMTLEPNKDLRPGDMVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLHCSFSPEPGFSLTQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDREGEGSKTALQPLKHSDSKEDDGQEIA

SEQ ID NO: 14 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDTTKRPVTTTKREVNSAI

SEQ ID NO: 16 - protein, 309 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTESWNLLLLLS

SEQ ID NO: 17 - protein, 534 aa, Homo sapiens; identical to SEQ ID NO: 11

SEQ ID NO: 18 - protein, 534 aa, Homo sapiens [sequence omitted]

SEQ ID NO: 19 - protein, 316 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 20 - protein, 463 aa, Artificial Sequence
  Fusion protein
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFLDLLAQGNASLRLQSVRVADEGSFTCFVSIRDFGSAAVSLQVAAPYSKPSMTLEPNKDLRPGDMVTITCSSYQGYPEAEVFWQDGQGVPLTGNVTTSQMANEQGLFDVHSILRVVLGANGTYSCLVRNPVLQQDAHSSVTITRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLS